(19) United States
(12) Patent Application Publication
(10) Pub. No.: US 2016/0008024 A1
(43) Pub. Date: Jan. 14, 2016

(54) RENAL DENERVATION WITH STAGED ASSESSMENT

(71) Applicant: Arapeen Medical, LLC, Jeremy Ranch, UT (US)

(72) Inventors: Allison PAYNE, Salt Lake City, UT (US); Dennis PARKER, Centerville, UT (US); Rock HADLEY, Centerville, UT (US); Nassir MARROUCHE, Park City, UT (US); David JENKINS, Salt Lake City, UT (US); Jesse CROWNE, Salt Lake City, UT (US)

(21) Appl. No.: 14/795,603

(22) Filed: Jul. 9, 2015

Related U.S. Application Data
(60) Provisional application No. 62/022,625, filed on Jul. 9, 2014.

(51) Int. Cl. A61B 17/32

(52) U.S. Cl. CPC A61B 17/320068

(57) ABSTRACT

Non-invasive, outcome-confirmed renal denervation procedures include assessments performed before, during, and after an ablation procedure. Stimulation operations can be performed by ultrasound application apart from ablation and/or heating operations to determine the presence and/or condition of a renal nerve in a targeted region. Consistent nerve ablation can be achieved (e.g., in a spontaneous hypertensive patient) by assessing the pathophysiology of renal denervation via (1) reduction of blood pressure and/or (2) kidney and serum norepinephrine concentration.

### RELATED APPLICATION

This application claims the priority benefit of U.S. Provisional Application No. 62/022,625, filed Jul. 9, 2014, the entirety of which is hereby incorporated herein by reference.

### FIELD

The subject technology relates to ablation treatment by ultrasound energy, including by ablation of renal nerves, and corresponding procedure assessment.

### BACKGROUND

Hypertension represents a critical health challenge for millions of people, affecting 74.5 million adults in the United States and costing approximately $76.6 billion when considering direct and indirect costs. Despite the availability of numerous pharmaceutical agents, roughly 40% of patients have uncontrolled hypertension. Since increased age and obesity are two of the most significant risk factors for hypertension, these numbers are expected to drastically increase making the treatment of hypertension a significant public health challenge. While there are many with uncontrolled hypertension, this is usually due to lack of patient adherence to the physician prescribed treatment, or inadequate treatment. However, approximately 10% of the patient population who are currently taking 3 medications or more continue to have persistent high blood pressure and are identified with resistant hypertension.

Kidneys play a major role in the chronic regulation of blood pressure, mainly through the regulation of sodium and water excretion. Renal sympathetic nerves are key in initiating and maintaining systemic hypertension and regulate several renal functions that are believed to contribute to hypertension including renal hemodynamics, renal tubular absorption of sodium and water, norepinephrine release and the renin secretion rate. Indeed, before effective pharmaceutical treatments were available, the surgical removal of these nerves was used as an effective treatment for hypertension, although this procedure had high morbidity rates. The proposed use of a non-invasive renal denervation procedure has the potential to produce the same efficacy without the high morbidity rates.

Many traditional renal denervation techniques apply energy with a catheter-based technique increasing procedural risk and restricting the eligibility of potential candidates. High intensity focused ultrasound (HIFU) is a completely non-invasive energy delivery technology that can deliver energy deep into tissue and can facilitate change on a cellular level through both thermal and mechanical effects. Additionally, nerve conduction can be temporarily or permanently suspended through application of HIFU. Applying HIFU under MRI guidance (MRgHIFU) provides accurate visualization of the treatment region and real-time monitoring of the energy delivery allowing for both treatment monitoring and efficacy assessment.

### SUMMARY

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clauses 1, 11-19, 29, 36, 45, 55, and 62. The other clauses can be presented in a similar manner.

Clause 1. A method of performing and assessing a renal nerve ablation procedure, comprising:
stimulating a region with a first ultrasound energy from an ultrasound device;
based on a physiological parameter induced by the first ultrasound energy, determining whether the region includes a target renal nerve;
if the region contains the renal nerve, heating the region with a second ultrasound energy from the ultrasound device;
stimulating the region with a third ultrasound energy from the ultrasound device; and
based on the physiological parameter to the third ultrasound energy, determining whether the renal nerve was ablated.

Clause 2. The method of clause 1, further comprising: if the region does not contain the renal nerve, stimulating a different region with fourth ultrasound energy from the ultrasound device.

Clause 3. The method of clause 1, further comprising:
measuring a first indicator of the physiological parameter prior to stimulating the region with the first ultrasound energy;
measuring a second indicator of the physiological parameter during and/or after the stimulating the region with the first ultrasound energy;
wherein determining whether the region includes the target renal nerve comprises comparing the first indicator to the second indicator.

Clause 4. The method of clause 1, further comprising:
measuring a first indicator of the physiological parameter during and/or after the stimulating the region with the first ultrasound energy;
measuring a second indicator of the physiological parameter during and/or after the stimulating the region with the third ultrasound energy;
wherein determining whether the renal nerve was ablated comprises comparing the first indicator to the third indicator.

Clause 5. The method of clause 1, wherein the physiological parameter comprises at least one of blood pressure, renal blood flow rate, or a concentration of medulla norepinephrine in an anatomy of the patient.

Clause 6. The method of clause 1, further comprising: if the renal nerve was ablated, stimulating a different region with fourth ultrasound energy from the ultrasound device.

Clause 7. The method of clause 1, wherein the first and/or third ultrasound energy satisfies an acoustic intensity threshold and/or a sonication pulse duration threshold necessary to achieve nerve stimulation.

Clause 8. The method of clause 1, wherein the first and/or third ultrasound energy does not satisfy an acoustic intensity threshold and/or a sonication pulse duration threshold necessary to achieve nerve ablation.

Clause 9. The method of clause 1, wherein the acoustic intensity threshold is 0.1-100 W/cm2 and the sonication pulse duration threshold is 5-250 ms.

Clause 10. The method of clause 1, wherein the second ultrasound energy satisfies an acoustic intensity threshold and/or a sonication pulse duration threshold necessary to achieve nerve ablation.

Clause 11. A system for performing and assessing a renal nerve ablation procedure, comprising:
a stimulation module configured to stimulate a region with a first ultrasound energy from an ultrasound device;
a determining module configured to determine whether the region includes a target renal nerve based on a physiological parameter induced by the first ultrasound energy;
a heating module configured to heat the region with a second ultrasound energy from the ultrasound device if the region contains the renal nerve;
wherein the stimulation module is further configured to stimulate the region with a third ultrasound energy from the ultrasound device; and
wherein the determining module is further configured to determine whether the renal nerve was ablated based on the physiological parameter to the third ultrasound energy.

Clause 12. A machine-readable medium comprising instructions for performing and assessing a renal nerve ablation method, the method comprising:
stimulating a region with a first ultrasound energy from an ultrasound device;
based on a physiological parameter induced by the first ultrasound energy, determining whether the region includes a target renal nerve;
if the region contains the renal nerve, heating the region with a second ultrasound energy from the ultrasound device;
stimulating the region with a third ultrasound energy from the ultrasound device; and
based on the physiological parameter to the third ultrasound energy, determining whether the renal nerve was ablated.

Clause 13. A method, comprising:
stimulating a region with a first ultrasound energy from an ultrasound device;
based on a physiological parameter induced by the first ultrasound energy, determining whether the region includes a target renal nerve;
if the region does not contain the renal nerve, stimulating a different region with second ultrasound energy from the ultrasound device.

Clause 14. A system for performing and assessing a renal nerve ablation procedure, comprising:
a stimulation module configured to stimulate a region with a first ultrasound energy from an ultrasound device;
a determining module configured to determine whether the region includes a target renal nerve based on a physiological parameter induced by the first ultrasound energy; and
an output module configured to output an indicator of whether the region includes the target renal nerve.

Clause 15. A machine-readable medium comprising instructions for performing and assessing a renal nerve ablation method, the method comprising:
stimulating a region with a first ultrasound energy from an ultrasound device;
based on a physiological parameter induced by the first ultrasound energy, determining whether the region includes a target renal nerve;
if the region does not contain the renal nerve, stimulating a different region with second ultrasound energy from the ultrasound device.

Clause 16. A method, comprising:
heating a region with a first ultrasound energy from an ultrasound device;
stimulating the region with a second ultrasound energy from the ultrasound device; and
based on a physiological parameter induced by the second ultrasound energy, determining whether a renal nerve in the region was ablated by the heating.

Clause 17. A system for performing and assessing a renal nerve ablation procedure, comprising:
a heating module configured to heat a region with a first ultrasound energy from an ultrasound device;
a stimulation module configured to stimulate the region with a second ultrasound energy from the ultrasound device; and
a determining module configured to determine whether a renal nerve in the region was ablated by the heating based on a physiological parameter induced by the first ultrasound energy; and
an output module configured to output an indicator of whether the region includes the target renal nerve.

Clause 18. A machine-readable medium comprising instructions for performing and assessing a renal nerve ablation method, the method comprising:
heating a region with a first ultrasound energy from an ultrasound device;
stimulating the region with a second ultrasound energy from the ultrasound device; and
based on a physiological parameter induced by the second ultrasound energy, determining whether a renal nerve in the region was ablated by the heating.

Clause 19. A method of performing and assessing a renal nerve ablation procedure, comprising:
stimulating a region with a first ultrasound energy from an ultrasound device;
based on an assessment, following initiation of the stimulating with the first ultrasound energy, of a physiological parameter affected by a target renal nerve, determining whether the region includes the target renal nerve;
when the region is determined to contain the target renal nerve, heating the region with a second ultrasound energy from the ultrasound device;
stimulating the region with a third ultrasound energy from the ultrasound device; and
based on an assessment, following initiation of the stimulating with the third ultrasound energy, of the physiological parameter, determining whether the target renal nerve was ablated.

Clause 20. The method of clause 19, further comprising, when the region is determined not to contain the target renal nerve, stimulating a different region with a fourth ultrasound energy from the ultrasound device.

Clause 21. The method of clause 19, further comprising:
measuring a first indicator of the physiological parameter prior to stimulating the region with the first ultrasound energy;
measuring a second indicator of the physiological parameter during and/or after the stimulating the region with the first ultrasound energy;
wherein determining whether the region includes the target renal nerve comprises comparing the first indicator to the second indicator.

Clause 22. The method of clause 19, further comprising:
measuring a first indicator of the physiological parameter during and/or after the stimulating the region with the first ultrasound energy;
measuring a second indicator of the physiological parameter during and/or after the stimulating the region with the third ultrasound energy;
wherein determining whether the target renal nerve was ablated comprises comparing the first indicator to the second indicator.

Clause 23. The method of clause 19, wherein the physiological parameter comprises blood pressure, renal blood flow rate, and/or a concentration of medulla norepinephrine in an anatomy of a patient.

Clause 24. The method of clause 19, further comprising, when the target renal nerve is determined to have been ablated, stimulating a different region with a fourth ultrasound energy from the ultrasound device.

Clause 25. The method of clause 19, wherein the first and/or third ultrasound energy satisfies an acoustic intensity threshold and/or a sonication pulse duration threshold necessary to achieve nerve stimulation.

Clause 27. The method of clause 25, wherein the acoustic intensity threshold is 0.1-100 W/cm2 and the sonication pulse duration threshold is 5-250 ms.

Clause 26. The method of clause 19, wherein the first and/or third ultrasound energy does not satisfy an acoustic intensity threshold and/or a sonication pulse duration threshold necessary to achieve nerve ablation.

Clause 28. The method of clause 19, wherein the second ultrasound energy satisfies an acoustic intensity threshold and/or a sonication pulse duration threshold necessary to achieve nerve ablation.

Clause 29. A method, comprising:
stimulating a region with a first ultrasound energy from an ultrasound device;
based on an assessment, following initiation of the stimulating with the first ultrasound energy, of a physiological parameter affected by a target renal nerve, determining whether the region includes the target renal nerve;
when the region is determined to contain the target renal nerve, heating the region with a second ultrasound energy from the ultrasound device;
when the region is determined not to contain the target renal nerve, stimulating a different region with a third ultrasound energy from the ultrasound device.

Clause 30. The method of clause 29, further comprising:
measuring a first indicator of the physiological parameter prior to stimulating the region with the first ultrasound energy;
measuring a second indicator of the physiological parameter during and/or after the stimulating the region with the first ultrasound energy;
wherein determining whether the region includes the target renal nerve comprises comparing the first indicator to the second indicator.

Clause 31. The method of clause 29, wherein the physiological parameter comprises blood pressure, renal blood flow rate, and/or a concentration of medulla norepinephrine in an anatomy of a patient.

Clause 32. The method of clause 29, wherein the first and/or third ultrasound energy satisfies an acoustic intensity threshold and/or a sonication pulse duration threshold necessary to achieve nerve stimulation.

Clause 33. The method of clause 32, wherein the acoustic intensity threshold is 0.1-100 W/cm2 and the sonication pulse duration threshold is 5-250 ms.

Clause 34. The method of clause 29, wherein the first and/or third ultrasound energy does not satisfy an acoustic intensity threshold and/or a sonication pulse duration threshold necessary to achieve nerve ablation.

Clause 35. The method of clause 29, wherein the second ultrasound energy satisfies an acoustic intensity threshold and/or a sonication pulse duration threshold necessary to achieve nerve ablation.

Clause 36. A method, comprising:
heating a region with a first ultrasound energy from an ultrasound device;
after the heating, stimulating the region with a second ultrasound energy from the ultrasound device; and
based on an assessment, following initiation of the stimulating with the second ultrasound energy, of a physiological parameter affected by a target renal nerve, determining whether a renal nerve in the region was ablated by the heating.

Clause 37. The method of clause 36, further comprising:
before heating the region, stimulating the region with a third ultrasound energy from the ultrasound device;
measuring a first indicator of the physiological parameter during and/or after the stimulating the region with the second ultrasound energy;
measuring a second indicator of the physiological parameter during and/or after the stimulating the region with the third ultrasound energy;
wherein determining whether the target renal nerve was ablated comprises comparing the first indicator to the second indicator.

Clause 38. The method of clause 36, further comprising, when the target renal nerve is determined to have been ablated, stimulating a different region with a third ultrasound energy from the ultrasound device.

Clause 39. The method of clause 36, further comprising, when the target renal nerve is determined not to have been ablated, further heating the region with a third ultrasound energy from the ultrasound device.

Clause 40. The method of clause 36, wherein the physiological parameter comprises blood pressure, renal blood flow rate, and/or a concentration of medulla norepinephrine in an anatomy of a patient.

Clause 41. The method of clause 36, wherein the second ultrasound energy satisfies an acoustic intensity threshold and/or a sonication pulse duration threshold necessary to achieve nerve stimulation.

Clause 42. The method of clause 41, wherein the acoustic intensity threshold is 0.1-100 W/cm2 and the sonication pulse duration threshold is 5-250 ms.

Clause 43. The method of clause 36, wherein the second ultrasound energy does not satisfy an acoustic intensity threshold and/or a sonication pulse duration threshold necessary to achieve nerve ablation.

Clause 44. The method of clause 36, wherein the first ultrasound energy satisfies an acoustic intensity threshold and/or a sonication pulse duration threshold necessary to achieve nerve ablation.

Clause 45. A system for performing and assessing a renal nerve ablation procedure, comprising:
a stimulation module configured to stimulate a region with a first ultrasound energy from an ultrasound device;
a determining module configured to determine whether the region includes a target renal nerve based on an assessment, following initiation of stimulating with the first ultrasound energy, of a physiological parameter affected by the target renal nerve; and
a heating module configured to heat the region with a second ultrasound energy from the ultrasound device when the region is determined to contain the target renal nerve;
wherein the stimulation module is further configured to stimulate the region with a third ultrasound energy from the ultrasound device; and
wherein the determining module is further configured to determine whether the target renal nerve was ablated based on an assessment, following initiation of stimulating with the third ultrasound energy, of the physiological parameter.

Clause 46. The system of clause 45, wherein the stimulation module is further configured to stimulate a different region with a fourth ultrasound energy from the ultrasound device when the region is determined not to contain the target renal nerve.

Clause 47. The system of clause 45, wherein the determining module is further configured to measure a first indicator of the physiological parameter prior to stimulating the region with the first ultrasound energy, measure a second indicator of the physiological parameter during and/or after the stimulating the region with the first ultrasound energy, and determine whether the region includes the target renal nerve by comparing the first indicator to the second indicator.

Clause 48. The system of clause 45, wherein the determining module is further configured to measure a first indicator of the physiological parameter during and/or after the stimulating the region with the first ultrasound energy, measure a second indicator of the physiological parameter during and/or after the stimulating the region with the third ultrasound energy, and determine whether the target renal nerve was ablated by comparing the first indicator to the second indicator.

Clause 49. The system of clause 45, wherein the physiological parameter comprises blood pressure, renal blood flow rate, and/or a concentration of medulla norepinephrine in an anatomy of a patient.

Clause 50. The system of clause 45, wherein the stimulation module is further configured to stimulate a different region with a fourth ultrasound energy from the ultrasound device when the target renal nerve is determined to have been ablated.

Clause 51. The system of clause 45, wherein the first and/or third ultrasound energy satisfies an acoustic intensity threshold and/or a sonication pulse duration threshold necessary to achieve nerve stimulation.

Clause 53. The system of clause 51, wherein the acoustic intensity threshold is 0.1-100 W/cm2 and the sonication pulse duration threshold is 5-250 ms.

Clause 52. The system of clause 45, wherein the first and/or third ultrasound energy does not satisfy an acoustic intensity threshold and/or a sonication pulse duration threshold necessary to achieve nerve ablation.

Clause 54. The system of clause 45, wherein the second ultrasound energy satisfies an acoustic intensity threshold and/or a sonication pulse duration threshold necessary to achieve nerve ablation.

Clause 55. A system for performing and assessing a renal nerve ablation procedure, comprising:
a stimulation module configured to stimulate a region with a first ultrasound energy from an ultrasound device;
a determining module configured to determine whether the region includes a target renal nerve based on an assessment, following initiation of stimulating with the first ultrasound energy, of a physiological parameter affected by the target renal nerve;
an output module configured to output an indicator of whether the region includes the target renal nerve; and
a heating module configured to heat the region with a second ultrasound energy from the ultrasound device when the region is determined to contain the target renal nerve;
wherein the stimulation module is further configured to stimulate a different region with a third ultrasound energy from the ultrasound device when the region is determined not to contain the target renal nerve.

Clause 56. The system of clause 55, wherein the determining module is further configured to measure a first indicator of the physiological parameter prior to stimulating the region with the first ultrasound energy, measure a second indicator of the physiological parameter during and/or after the stimulating the region with the first ultrasound energy, and determine whether the region includes the target renal nerve by comparing the first indicator to the second indicator.

Clause 57. The system of clause 55, wherein the physiological parameter comprises blood pressure, renal blood flow rate, and/or a concentration of medulla norepinephrine in an anatomy of a patient.

Clause 58. The system of clause 55, wherein the first and/or third ultrasound energy satisfies an acoustic intensity threshold and/or a sonication pulse duration threshold necessary to achieve nerve stimulation.

Clause 59. The system of clause 58, wherein the acoustic intensity threshold is 0.1-100 W/cm2 and the sonication pulse duration threshold is 5-250 ms.

Clause 60. The method of clause 55, wherein the first and/or third ultrasound energy does not satisfy an acoustic intensity threshold and/or a sonication pulse duration threshold necessary to achieve nerve ablation.

Clause 61. The system of clause 55, wherein the second ultrasound energy satisfies an acoustic intensity threshold and/or a sonication pulse duration threshold necessary to achieve nerve ablation.

Clause 62. A system for performing and assessing a renal nerve ablation procedure, comprising:
a heating module configured to heat a region with a first ultrasound energy from an ultrasound device;
a stimulation module configured to stimulate the region with a second ultrasound energy from the ultrasound device;
a determining module configured to determine whether a renal nerve in the region was ablated by the heating based on an assessment, following initiation of stimulating with the second ultrasound energy, of a physiological parameter affected by a target renal nerve; and
an output module configured to output an indicator of whether the region includes the target renal nerve.

Clause 63. The system of clause 62, wherein the stimulation module is further configured to stimulate the region with a third ultrasound energy from the ultrasound device before heating the region; and wherein the determining module is further configured to measure a first indicator of the physiological parameter during and/or after the stimulating the region with the second ultrasound energy, measure a second indicator of the physiological parameter during and/or after the stimulating the region with the third ultrasound energy, and determine whether the target renal nerve was ablated by comparing the first indicator to the second indicator.

Clause 64. The system of clause 62, wherein the stimulation module is further configured to stimulate a different region with a third ultrasound energy from the ultrasound device when the target renal nerve is determined to have been ablated.

Clause 65. The system of clause 62, wherein the heating module is further configured to heat the region with a third ultrasound energy from the ultrasound device when the target renal nerve is determined not to have been ablated.

Clause 66. The system of clause 62, wherein the physiological parameter comprises blood pressure, renal blood flow rate, and/or a concentration of medulla norepinephrine in an anatomy of a patient.

Clause 67. The system of clause 62, wherein the second ultrasound energy satisfies an acoustic intensity threshold and/or a sonication pulse duration threshold necessary to achieve nerve stimulation.

Clause 68. The system of clause 67, wherein the acoustic intensity threshold is 0.1-100 W/cm2 and the sonication pulse duration threshold is 5-250 ms.

Clause 69. The system of clause 62, wherein the second ultrasound energy does not satisfy an acoustic intensity threshold and/or a sonication pulse duration threshold necessary to achieve nerve ablation.

Clause 70. The system of clause 62, wherein the first ultrasound energy satisfies an acoustic intensity threshold and/or a sonication pulse duration threshold necessary to achieve nerve ablation.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 shows a view of portions of a patient's anatomy.

FIG. 2 shows a view of portions of a patient's anatomy.

FIG. 3 shows a view of an array of transducers focused on a renal nerve (not to scale), according to some embodiments of the present disclosure, according to some embodiments of the subject technology.

FIG. 4 shows a view of portions of a patient's anatomy that can be targeted during a therapeutic procedure, according to some embodiments of the subject technology.

FIG. 5 shows a pre-treatment assessment procedure for a target that can be applied to each renal artery, according to some embodiments of the subject technology. Stimulation pulses can be applied at Iₜₕᵣₑₛₕ and tₜₕᵣₑₛₕ. Blood pressure (“BP”) and renal blood flow (“RBF”) are measured with the decision metric and expected outcomes as discussed herein.

FIG. 6 shows a renal denervation ablation procedure that can be applied to each renal artery, according to some embodiments of the subject technology. Blood pressure (“BP”) and renal blood flow (“RBF”) are measured with the decision metric and expected outcomes as discussed herein.

FIG. 7 shows a post-treatment outcome assessment procedure that can be applied to each renal artery, according to some embodiments of the subject technology. Stimulation pulses can be applied at Iₜₕᵣₑₛₕ and tₜₕᵣₑₛₕ. Blood pressure (“BP”) and renal blood flow (“RBF”) are measured with the decision metric and expected outcomes as discussed herein.

FIG. 8 shows a renal denervation ablation procedure with pre-treatment and post-treatment assessment procedures that can be applied to each renal artery, according to some embodiments of the subject technology. Stimulation pulses can be applied at Iₜₕᵣₑₛₕ and tₜₕᵣₑₛₕ. Blood pressure (“BP”) and renal blood flow (“RBF”) are measured with the decision metric and expected outcomes as discussed herein.

FIGS. 9A, 9B, and 9C show vascular phantom constructions. FIG. 9A shows a photo of a vascular phantom mold with excised rabbit aorta and fiber optic temperature probe in place. FIG. 9B shows a photo of the same vascular phantom after gelatin was poured around the vessel. FIG. 9C shows a photo of a fiber optic temperature probe used in both the phantom and in vivo pig experiments.

FIGS. 10A and 10B show a sonication pattern in the vascular phantom. FIG. 10A shows an axial MR image of gelatin vascular phantom placed over focused ultrasound transducer. Three planes of a nine-point raster pattern were sonicated centered around the vessel. FIG. 10B shows a top view of a single nine-point raster pattern. The approximate location of the vessel is shown by the dashed lines. The approximate location of tip of the fiber optic probe is indicated by the green star. Spacing between the points in plane and between planes was 1 cm in one example.

FIG. 11 shows an axial T1w image of a rat placed in an oblique supine position on the MRgHIFU device. The approximate volume that can be monitored in real-time with MR thermometry is shown (dashed box).

FIG. 12A shows a schematic of pig placement on MRgFUS device. The position of the transducer below the animal with the cone (green) depicting the ultrasound focus and positioning of the nine RF receiver coils are seen.

FIG. 12B shows an axial T1w image from a swine model. The acoustic window (dashed lines) and 6 ablation points are shown (white ovals) surrounding the right renal artery. The images used for targeting show important detail including the spinous process (solid triangle), aorta (dashed arrow) and bowel (hollow triangle).

FIG. 12C shows an axial T1-map of a pig immediately after a unilateral RD procedure. HIFU sonications were applied around the right renal artery resulting in a temporary decrease in blood flow to the right kidney, indirectly measured by the T1 in the kidney. The average T1 in the right kidney (solid arrow, T1=1000 ms) was lower than the T1 in the left kidney (hollow arrow, T1=1450 ms).

FIG. 13A shows a chart demonstrating vascular phantom thermal response.

Peak fiber optic temperature change measured in the vascular gelatin phantom during each sonication as a function of distance between the focused ultrasound beam location and fiber optic probe tip. The two tested flow rates, 80 mL/min (“x”) and 40 mL/min (“∘”) are shown.

FIG. 13B shows a chart demonstrating porcine model thermal response. Peak fiber optic temperature change measured during the RSD procedure in each of the five animals. Decreasing trends of temperature rise as a function of distance from the fiber optic probe tip to the focal spot position was observed in all animals.

FIG. 14A shows a coronal view of a plane in the near field of the ultrasound beam for animal 3. The enlarged inset indicates an area that accumulated thermal dose with potential necrotic damage. The total volume with potential damage in this animal was 123 mm³. The values for all animals are given in Table 3.

FIG. 14B shows an enhancement due to heating denoted by box (enlarged inset) seen around the spinous process in a post-procedure delayed contrast-enhanced T1w image.

FIG. 15A shows H&E stained sections of a treated artery in animal 5. FIG. 15B shows H&E stained sections of a control artery in animal 5. Inset (N) indicates the arterial nerves. Nerves damage is present in the treated side as exhibited by perineural fibrosis (arrow) and degradation of the nerve fibers (asterisk). There was no apparent damage to either of the vessels (V).

FIG. 16 shows a block diagram of a MRI-guided ultrasound system, according to some embodiments of the subject technology.

FIG. 17 shows a block diagram of a MRI-guided ultrasound system, according to some embodiments of the subject technology.

FIG. 18 shows a block diagram of a MRI-guided ultrasound system, according to some embodiments of the subject technology.

### DETAILED DESCRIPTION

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology.

According to some embodiments, an apparatus and method for using MRI-guided focused ultrasound to ablate sympathetic nerves near the renal arteries may be employed to allow reduction of blood pressure. According to some embodiments, provided are devices and procedures to focus high intensity, ultrasonic acoustic waves into the tissue. High-intensity focused ultrasound (“HIFU”) is a highly precise medical procedure using high-intensity focused ultrasound to heat and destroy tissue.

As an acoustic wave propagates through the tissue, at least part of it is absorbed and converted to heat. With focused beams, a very small focus can be achieved deep in tissues. When hot enough, the tissue is thermally coagulated. By focusing at more than one place or by scanning the focus, a volume of tissue can be thermally ablated. In HIFU therapy, ultrasound beams are focused on targeted tissue, and due to the significant energy deposition at the focus, temperature within the tissue rises, destroying the diseased tissue by coagulation necrosis. Each sonication of the beams treats a precisely defined portion of the targeted tissue.

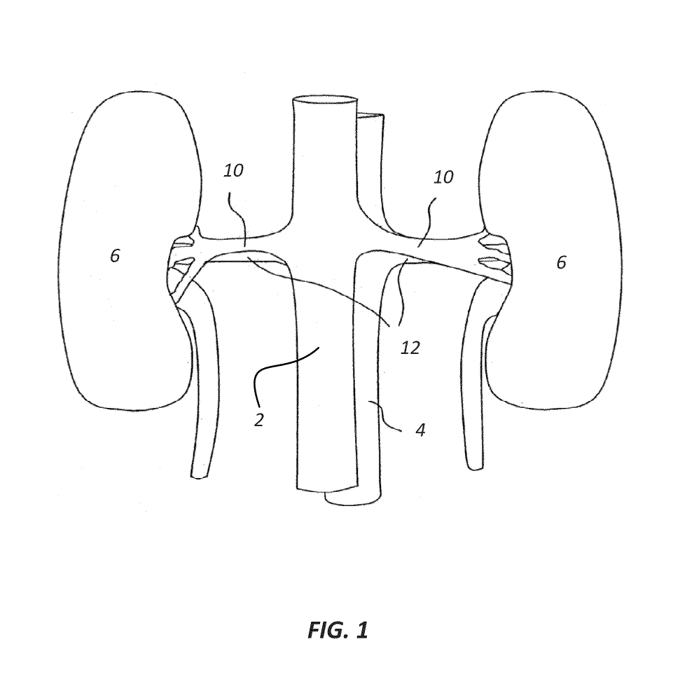

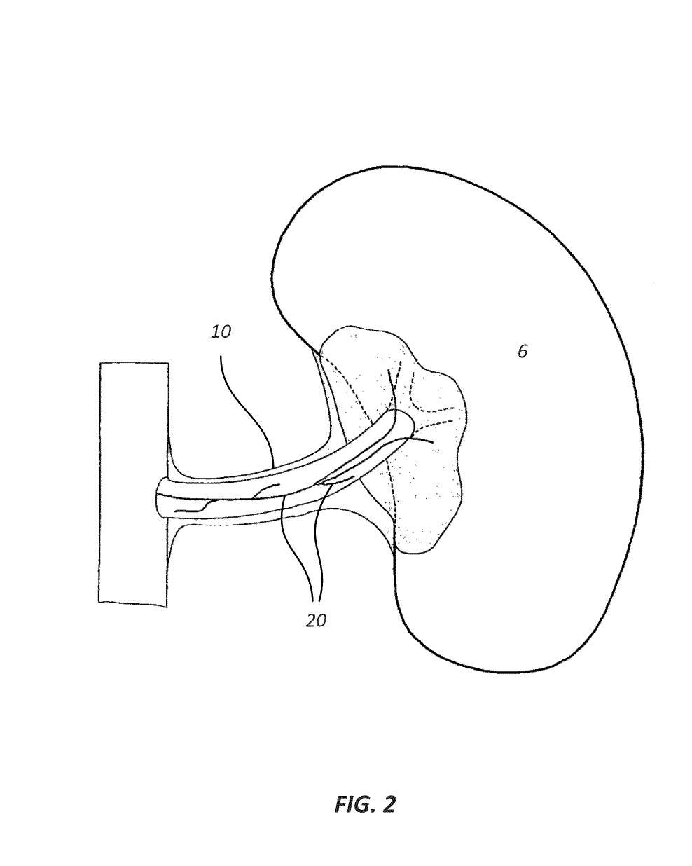

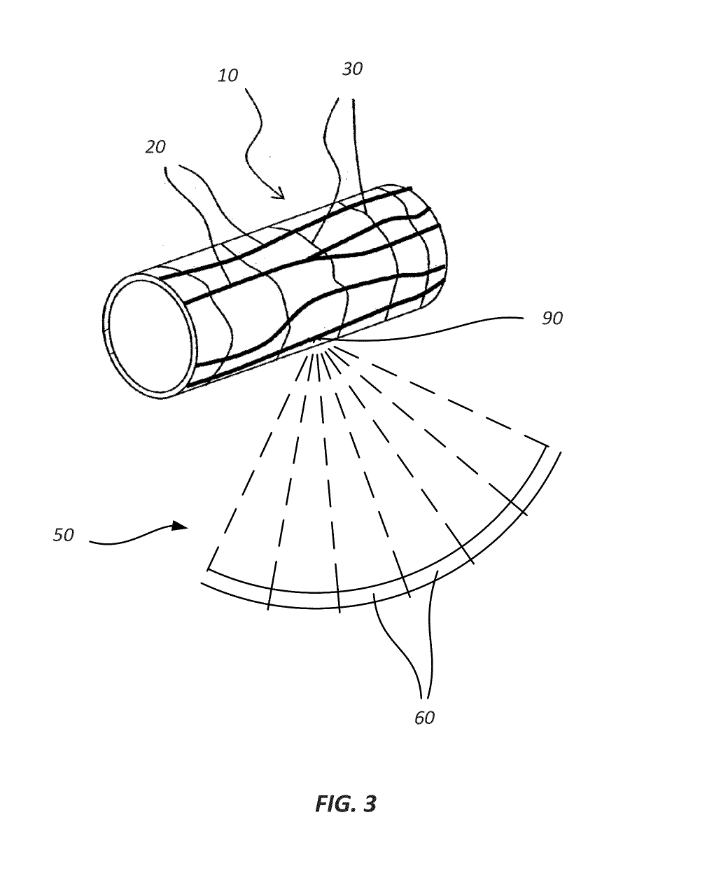

With reference now to FIG. 1, the human renal anatomy includes kidneys 6 that are supplied with oxygenated blood by renal arteries 10, which are connected to the heart by the abdominal aorta 2. Deoxygenated blood flows from the kidneys to the heart via renal veins 12 and the inferior vena cava 4. FIGS. 2-3 illustrate portions of the renal anatomy, including renal nerves 20 extending longitudinally along a lengthwise dimension of the renal artery 10 generally within the adventitia of the artery. The renal artery 10 has smooth muscle cells 30 that surround the arterial circumference and spiral around the artery.

According to some embodiments, as shown in FIG. 3, a HIFU device 50 may comprise one or more transducers 60 (e.g., 60a and 60b) for emitting ultrasound energy. Where a HIFU device 50 comprises an array of transducers 60 is provided, constituent transducers 60 of the array may be directed to converge generally at a focal region 90. The focal region 90 may be determined by position of the constituent transducers 60 relative to each other or position of the array relative to a target site.

According to some embodiments, as shown in FIG. 4, a HIFU device 50 can target tissue within one or both of two lateral sides of the patient. Transducers 60 of a HIFU device 50 can transmit sonic energy through one or both of two windows 94 (e.g., 94a and 94b) to focus on the target site. According to some embodiments, windows 94 can be located on an anterior side of the patient (e.g., with the patient in supine position) or a posterior side of the patient (e.g., with the patient in proposition). A procedure involving a HIFU device 50 can access one or more target sites (e.g., of the renal artery 10) through windows 94 located on a left anterior side of the patient, a right anterior side of the patient, a left posterior side of the patient, and/or a right posterior side of the patient. Target sites can be accessed through any combination of such windows 94 in sequence and/or simultaneously. For example, as shown in FIG. 4, a HIFU device 50 can access regions at or near one or more renal arteries 10 through a window 94 above a pelvis 16 of a patient and below ribs 14 of the patient.

Remote, localized tissue ablation using HIFU can include sudden thermal necrosis due mainly to the absorption of ultrasound energy. The temperatures thus induced (e.g., about 60-80° C.) can produce irreversible changes in the targets. Target temperature thresholds may be any temperature above body temperature. For example, target temperature thresholds may include temperatures equal to or greater than 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C.

Therapeutic ultrasound may be provided as minimally invasive or non-invasive. Further, it may be provided transcutaneously, subcutaneously, intravascularly, inter alia. In addition to the above, an ultrasound beam can also be focused geometrically, for example with a lens, with a spherically curved transducer, or electronically, by adjusting the relative phases of elements in an array of transducers (a “phased array”). By dynamically adjusting the electronic signals to the elements of a phased array, the beam can be steered to different locations, and aberrations due to tissue structures can be corrected.

Incorporated herein by reference are the following US patents and/or publications containing further teachings regarding HIFU therapy: US Pub. No. 2007/0167773, published on Jul. 19, 2007; U.S. Pat. No. 5,769,790, issued on Jun. 23, 1998; US Pub. No. 2008/0312561, published on Dec. 18, 2008.

Magnetic resonance guided high intensity focused ultrasound (“MRgHIFU”) nerve stimulation can be used to acutely assess procedure success. A combination of HIFU parameters can result in successful and repeatable renal denervation as measured by one or both of primary outcomes: (1) a decrease of blood pressure and kidney and/or (2) blood norepinephrine concentration. The effects of MRgHIFU on the renal sympathetic nerves can be quantified through careful cataloguing and histomorphometric analysis of the nerves.

According to some embodiments, MRgHIFU is used to stimulate the renal sympathetic nerves to attempt to change a physiological parameter of the patient, such as an increase of blood pressure and/or a reduction of renal blood flow allowing, for example, which may provide an acute end-point assessment of the renal denervation procedure. The physiological parameter can indicate or represent a condition of the patient or a portion of the patient. The physiological parameter can be qualified or characterized by an amount, magnitude, quantity, rate, level, or other measurable aspect. The physiological parameter can be affected by a nerve or other tissue targeted, or attempted to be targeted, in stimulation and/or ablation procedures. For example, stimulation and/or ablation can attempt to alter the function of a targeted tissue such that a change in the physiological parameter is intended. Whether the change has occurred can be determined by measuring or otherwise observing the physiological parameter during and/or after the stimulation and/or ablation is performed. The verification can further include measuring or otherwise observing the characteristic before the stimulation and/or ablation is performed and making a comparison of the measurements or observations.

In an exemplary method of the subject technology, the HIFU sonication parameters that will result in sympathetic renal nerve stimulation in a pre-clinical spontaneous hypertensive patient can be determined, as assessed by a combination of invasive blood pressure measurements and renal blood flow measured with MRI techniques. Further, the pathophysiological status of the nerves can be characterized based on histomorphometric analysis of the nerves after the stimulation procedure.

According to some embodiments, a combination of HIFU parameters can cause a consistent and repeatable denervation effect to the renal sympathetic nerves resulting in a reduction in blood pressure and kidney norepinephrine levels without measurable collateral damage to normal tissues. In an exemplary method of the subject technology, a bilateral renal denervation is performed in a pre-clinical spontaneous hypertensive patient using a range of MRgHIFU intensity values. Resting pre- and post-procedure blood pressure and serum norepinephrine measurements can be evaluated at several time points and subsequently compared to predetermined expected outcomes (e.g., relative to a control group). Norepinephrine concentration in the kidney tissue can be evaluated based on expected outcomes. The location, density, area and physiological status of nerves along the renal artery can be quantified through histological analysis. The potential for evaluating the successful denervation of an identified target location can be assessed through interleaving nerve stimulation pulses with ablative HIFU sonications.

The quantified information regarding the physiological response of renal denervation can contribute to determination of an endpoint for renal denervation using a completely non-invasive technology. Such a determination can be applied in an MRgHIFU procedure for outcome-confirmed renal denervation for control of resistant hypertension.

A reduction of blood pressure at various time points is the primary outcome used for assessment in most clinical trials, but the decrease in blood pressure may not occur until 30 days post procedure. Limited studies have evaluated secondary measures that complement the blood pressure endpoint. It was shown that renal denervation results in the reduction of muscle sympathetic nerve activity, sustained for up to one year post-renal denervation procedure. Significant reduction of the norepinephrine spillover accompanying the decrease in blood pressure has been observed. While these secondary outcomes are assessed on a limited basis, they are currently not applied in all renal denervation procedures. The addition of clinically viable endpoints at the time of the procedure would greatly improve the potential of this treatment approach.

MRgHIFU allows for accurate delineation of the treatment target, real-time treatment feedback with thermometry maps or other MR images and post-treatment assessment. Unlike other commonly used image-guided minimally invasive thermal therapy procedures such as RF-, laser- and cryo-ablation, MRgHIFU is completely non-invasive. This feature provides several benefits when compared to traditional surgery, including shorter recovery time, lowered risk of infection and reduced anesthesia requirements. Clinically, MRgHIFU is currently utilized to treat numerous types of cancers, neurological disorders, provide localized delivery of drugs, open the blood brain barrier, and affect nerve functionality.

MRgHIFU can non-invasively treat hypertension through renal denervation, offering several advantages over catheter-based techniques. Renal denervation has been performed both pre-clinically and clinically with ultrasound-guided HIFU. Performing the procedure under MR guidance can increase both the safety and efficacy of the procedure, as well as provide a mechanism to monitor treatment efficacy at the time of the procedure. Currently, catheter-based techniques are done under fluoroscopic guidance. The only feedback provided to the clinician is probe impedance readings (RF ablation), indicating whether appropriate contact has been made with the vessel wall. In contrast, performing renal denervation under MR guidance would allow the clinician complete control over the entire procedure. Treatment planning, real-time procedure monitoring and treatment end-point assessment could all potentially be accomplished. In addition, treating resistant hypertension non-invasively through renal denervation with MRgHIFU would potentially allow the treatment of a greater number of patients when compared to the catheter-based techniques, since anatomical variations would not exclude patients from the procedure.

HIFU can reduce or stop nerve function, but the application of particular combinations of ultrasound parameters can result in nerve stimulation. High frequency, short HIFU bursts (˜500 W/cm²) increased the excitability of myelinated nerves without any significant temperature increase (<0.5° C.). Increased action potential, conduction velocity, and amplitude has been demonstrated in vitro. Transcranical neuromodulation is possible, and seizure suppression and eye abduction have been demonstrated in rat models. The ultrasound parameters necessary for successful transcranial neurostimulation have been demonstrated, and different neural circuits can be activated based on the location of the HIFU focus. In addition, it has also been shown that HIFU can also induce somatic and auditory sensations.

Catheter-based and extracorpeal devices can bring about significant decreases in both systolic and diastolic blood pressure. However, a better understand of the physiology involved and the mechanism behind the blood pressure reduction can better guide procedures. An outcome-confirmed metric can assist with evaluations both during and immediately after a procedure.

According to some embodiments, the subject technology includes a non-invasive, outcome-confirmed renal denervation procedure with an acute end-point assessment. Based on particular MRgHIFU parameters, consistent nerve ablation can be achieved in a spontaneous hypertensive patient by assessing the pathophysiology of renal denervation via (1) reduction of blood pressure and/or (2) kidney and serum norepinephrine concentration.

According to some embodiments, the HIFU parameters necessary to achieve peripheral nerve stimulation can include acoustic intensity threshold (Iₜₕᵣₑₛₕ), sonication pulse duration (tₜₕᵣₑₛₕ), pulse repetition frequency, number of pulses, and/or a total sonication procedure duration.

According to some embodiments, an Iₜₕᵣₑₛₕ necessary to achieve peripheral nerve stimulation can be in the range of 0.1-100 W/cm². For example, tₜₕᵣₑₛₕ can be about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 20.0, 30.0, 40.0, 50.0, 60.0, 70.0, 80.0, 90.0, or 100.0 W/cm². By further example, Iₜₕᵣₑₛₕ can be within a range defined by any two of the above values.

According to some embodiments, an Iₜₕᵣₑₛₕ necessary to achieve peripheral nerve stimulation can be determined by evaluating effectiveness through invasive or non-invasive measurements of one or more physiological parameters such as, for example, blood pressure and MRI measurements of renal blood flow. For example, after a patient is positioned to target the renal artery (side chosen arbitrarily), an ultrasound beam can be focused at an area adjacent to the artery close to the renal pelvis. The acoustic intensity can be incrementally varied (e.g., increased). According to some embodiments, a sonication time of 50 ms can be applied at a pulse repetition frequency of 2 Hz for 2 seconds at each intensity value. The entire acoustic intensity interval range can be applied bilaterally. The stimulation procedure can be monitored in real-time using a 3D segmented-EPI MR thermometry sequence. Baseline blood pressure, norepinephrine spillover, and renal blood flow in both kidneys can be assessed pre-stimulation procedure. While the blood pressure can be continuously monitored during the entire stimulation procedure, the renal blood flow can be assessed after each stimulation pulse. Post-procedure blood pressure and norepinephrine spillover can be obtained every 5 days post-procedure and the norepinephrine kidney concentration can be obtained 30-days post-procedure. Blood pressure and renal blood flow can be evaluated as a function of intensity for each animal. Iₜₕᵣₑₛₕ can be defined as the stimulus that elicits a minimum of an increase in blood pressure and/or a decrease in renal blood flow. The increase in blood pressure and/or the decrease in renal blood flow can be by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or greater than 30%. The determined tₜₕᵣₑₛₕ can be applied in subsequent nerve stimulation operations.

According to some embodiments, a sonication pulse duration (tₜₕᵣₑₛₕ) necessary to achieve peripheral nerve stimulation can be in the range of 5-250 ms. For example, tₜₕᵣₑₛₕ can be about 5, 10, 20, 30, 40, 50, 60, 70, 80, 90, 100, 110, 120, 130, 140, 150, 160, 170, 180, 190, 200, 210, 220, 230, 240, or 250 ms. By further example, tₜₕᵣₑₛₕ can be within a range defined by any two of the above values.

According to some embodiments, a tₜₕᵣₑₛₕ necessary to achieve peripheral nerve stimulation can be determined by evaluating effectiveness through invasive or non-invasive measurements of blood pressure and MRI measurements of renal blood flow. For example, after Iₜₕᵣₑₛₕ has been identified, the sonication time can be incrementally varied (e.g., increased) within a range of 5-250 ms at a pulse repetition rate of 2 Hz for 2 seconds in order to determine the most effective sonication pulse duration (tₜₕᵣₑₛₕ) for nerve stimulation as assessed by the blood pressure and renal blood flow stimulus response. The blood pressure and renal blood flow as a function of sonication pulse duration can be evaluated. Sonication pulse duration tₜₕᵣₑₛₕ can be defined as the parameter that elicits a larger or the largest deviation from baseline of both blood pressure and renal blood flow.

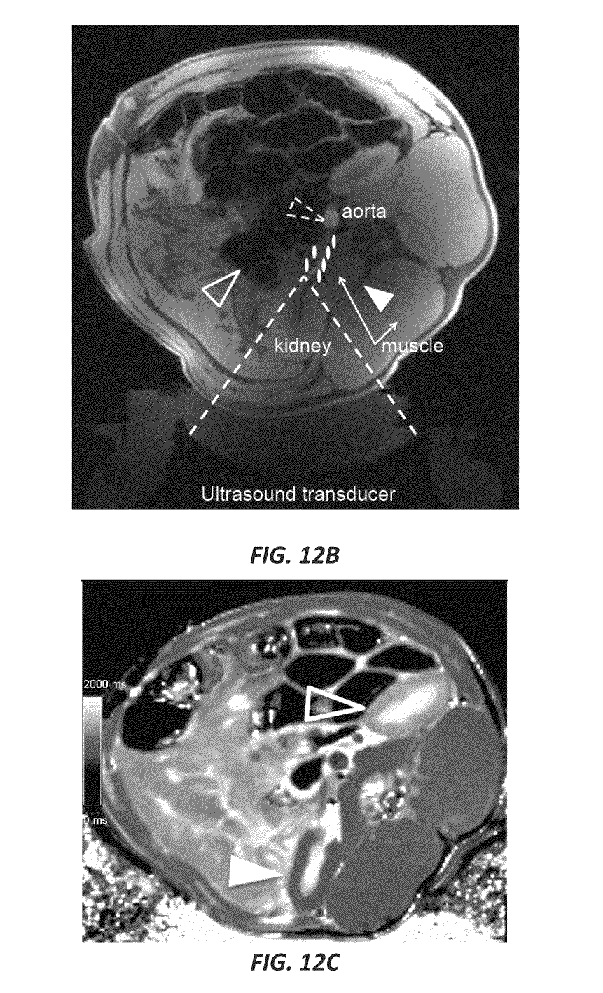

According to some embodiments, stimulation effectiveness can be evaluated by various methods. For example, an MR-compatible invasive blood pressure system (SA Instruments, Inc.) can be used to continuously monitor the blood pressure of the patient during the entire MRgHIFU stimulation protocol. By further example, the renal blood flow can be assessed after each stimulation attempt using arterial spin labeling (ASL) techniques, by obtaining T1 (ECG-triggered Modified Look-Locker Inversion Recovery sequence), and/or T2 (T2-prepared TrueFISP sequence) maps to indirectly assess renal blood flow. Spatial resolution and coverage for multiple imaging techniques can be compared and reconciled. Changes in both T1 (see FIG. 12C) and T2 values in the kidney have been measured on the treated side after a unilateral renal denervation procedure in a swine model. While the stimulation procedure may not have as large of an effect as renal denervation, a detectable decrease in renal blood flow can be detectable. Patients can be monitored for many (e.g., 30) days post-stimulation procedure with blood pressure and venous blood draws for serum norepinephrine measurements obtained every several (e.g., 5) days.

According to some embodiments, Iₜₕᵣₑₛₕ and tₜₕᵣₑₛₕ can be selected or determined to (1) effectively and sufficiently stimulate the nerves bilaterally rally and (2) avoid or reduce any nerve damage. The stimulation procedure using Iₜₕᵣₑₛₕ and tₜₕᵣₑₛₕ discussed herein can be repeated multiple times. Blood pressure, norepinephrine spillover, and renal blood flow can be obtained and analyzed as discussed herein. Nerve histomorphometric analysis can also be performed 30 days post-procedure in order to assess the nerve physiological status.

According to some embodiments, pre- and post-procedure blood pressure and norepinephrine spillover can be compared to kidney norepinephrine concentration and nerve area in non-treated control patients. Statistical significance can be set at p<0.05.

Stimulation may create a transient effect on blood pressure and/or renal blood flow. Since the blood pressure can be continuously monitored, transient changes can be detected while the renal blood flow measurements can be discrete. For long measurement times, measurements of renal blood flow can be accelerated with an undersampled acquisition to reconstruct the images with a constrained reconstruction algorithm. Furthermore, the contralateral kidney may counteract any stimulation effect on the opposing side. Applying multiple stimulation pulses may also cause edema around the renal artery causing reduced renal blood flow. The potential edema can be evaluated using the pre- and post-procedure imaging and the number of stimulation pulses can be reduced if necessary.

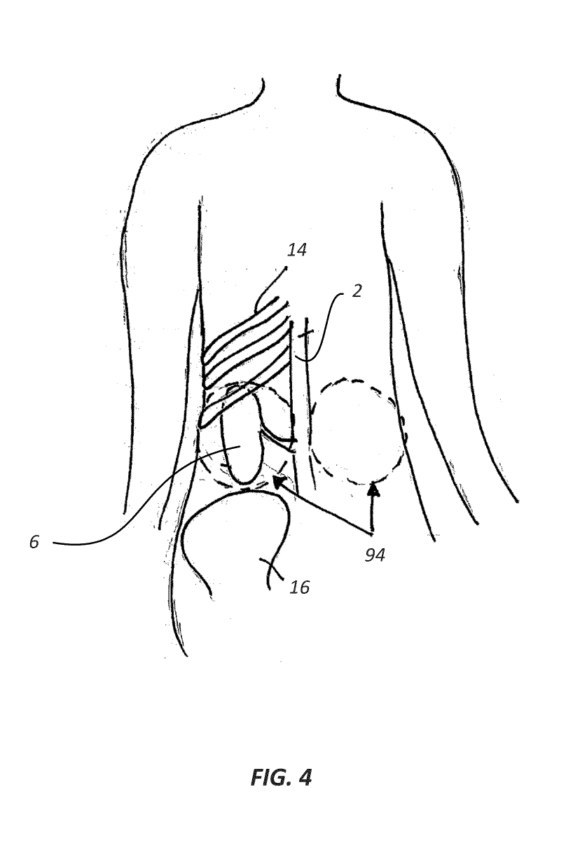

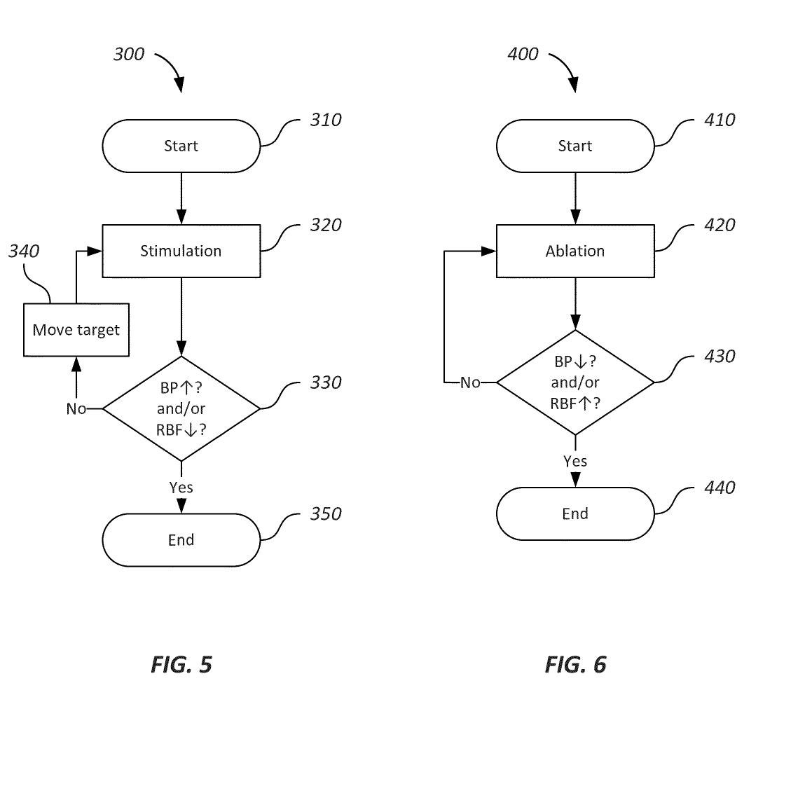

The protocols shown in FIGS. 5-8 can be applied bilaterally adjacent to each renal artery. Baseline blood pressure, blood samples and renal blood flow measurements in both kidneys can be obtained pre- and post-procedure. Blood samples and resting blood pressure measurements can be obtained every 5 days post-procedure. The effectiveness of using the stimulation protocol to predict successful renal denervation can be measured by comparing all three blood pressure and renal blood flow measurements obtained during the procedure. The long-term outcome of the renal denervation procedure can be evaluated using blood pressure and norepinephrine kidney concentration as the primary outcomes.

According to some embodiments, stimulation and or ablation can be achieved by one or a plurality of various methods, means, and mechanisms.

For example, Transcutaneous Electrical Nerve Stimulation (“TENS”) can provide non-invasive (skin surface) electricalstimulation. According to some embodiments, a microcurrent TENS unit can use a unique wave form. The current can be from 250 microamps up to about 900 microamps with a peak current of six milliamps. The current can be applied through a pair of electrodes in the form of high-frequency monophasic bursts of a direct current with a carrier signal from around 10,000 Hz to 19,000 Hz. The signal can be modulated at a relatively lower frequency (0.3 Hz up to 10,000 Hz). These modulated carrier signals can be from about 0.05 seconds to 10 seconds in duration. The electrodes can be reversed as simulating a biphasic form yet the character is a monophasic DC signal. According to some embodiments, electrodes can apply a constant direct current of 100-300 microamps for approximately 1-20 minutes.

Incorporated herein by reference are the following US patents and/or publications containing further teachings regarding TENS: U.S. Pat. No. 4,989,605, published on Feb. 5, 1991; U.S. Pat. No. 5,522,864, published on Jun. 4, 1996; U.S. Pat. No. 6,275,735, published on Aug. 14, 2001.

By further example, Pulsed Electromagnetic Field (“PEMF”) therapy can provide electro stimulation and/or electrical modulation (e.g., ablation). Pulsed electromagnetic fields are low-energy, time-varying magnetic fields that can be used to treat therapeutically resistant problems of the musculoskeletal system. Those problems include spinal fusion, ununited fractures, failed arthrodeses, osteonecrosis, and chronic refractory tendonitis, decubitus ulcers and ligament and tendon injuries. PEMF therapy can use one or more transducers to provide PEMF therapeutic stimulation to a target area.

Incorporated herein by reference are the following US patents and/or publications containing further teachings regarding PEMF: U.S. Pat. No. 7,783,348, published on Aug. 24, 2010; U.S. Pat. No. 5,181,902, published on Jan. 26, 1993.

According to some embodiments, focused or unfocused ultrasound, TNES, PEMF, cooling, cryogenic, pulsed RF, thermal RF, thermal, or non-thermal microwave, thermal or non-thermal DC, as well as any combination thereof, may be employed to stimulate or denervate.

According to some embodiments, as shown in FIG. 5, a procedure 300 may start at operation 310, optionally following a different procedure. In operation 320, a target region is stimulated, and a physiological response is verified in operation 330. If no physiological response occurs (e.g., increase of blood pressure, reduction of renal blood flow, etc.), the target is moved in operation 340 and a new stimulation procedure is commenced. If a physiological of response does occur, then the procedure 300 can end in operation 350, optionally leading into another procedure.

According to some embodiments, as shown in FIG. 6, a procedure 400 may start at operation 410, optionally following a different procedure. In operation 420, a target region is ablated, and a physiological response is verified in operation 430. If no physiological response occurs (e.g., decrease of blood pressure, increase of renal blood flow, etc.), the ablation can continue. If a physiological of response does occur, then the procedure 400 can end in operation 450, optionally leading into another procedure.

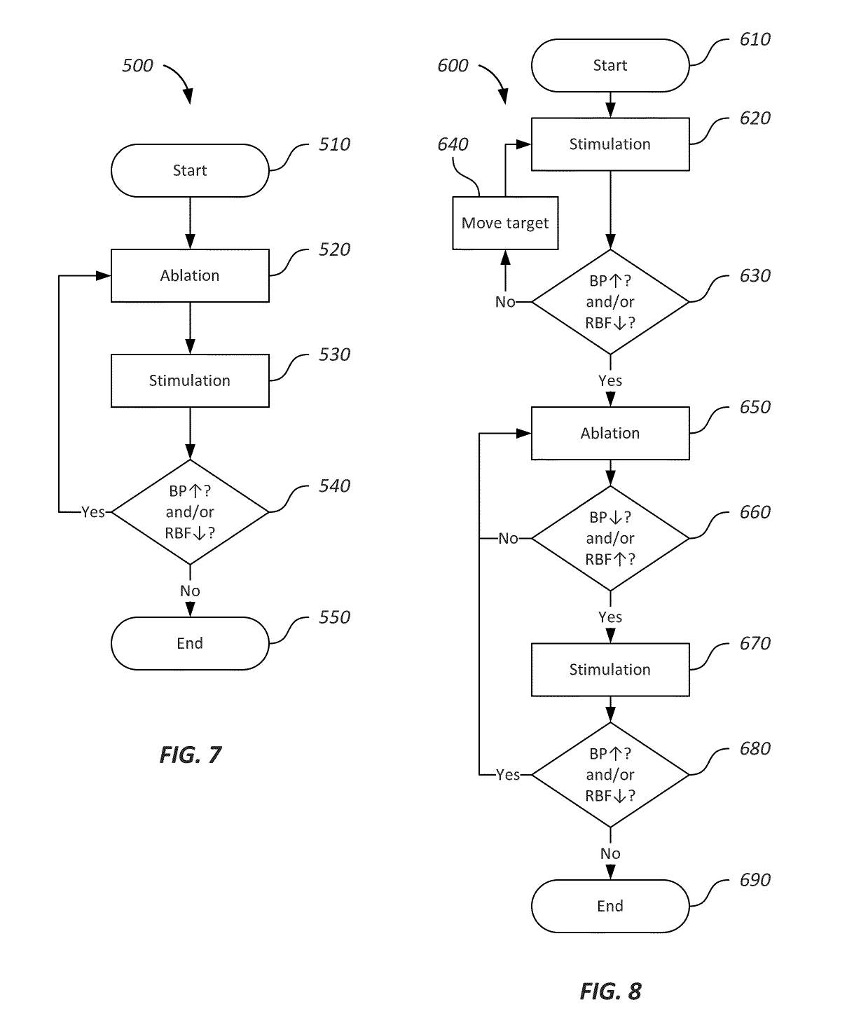

According to some embodiments, as shown in FIG. 7, a procedure 500 may start at operation 510, optionally following a different procedure. In operation 520, a target region is ablated. In operation 530, the target region is stimulated, and a physiological response is measured in operation 540. If no physiological response occurs (e.g., increase of blood pressure, reduction of renal blood flow, etc. not exceeding a predetermined threshold), then the nerve in the target region is determined to be sufficiently ablated, and the procedure 500 can end in operation 550, optionally leading into another procedure (e.g., moving to another target region). If a physiological of response does occur (e.g., exceeding a predetermined threshold), then the nerve in the target region can be determined to be insufficiently ablated and further ablation in operation 520 can commence or be resumed.

According to some embodiments, as shown in FIG. 8, a procedure 600 may start at operation 610, optionally following a different procedure. In operation 620, a target region is stimulated, and a physiological response is verified in operation 630. If no physiological response occurs (e.g., increase of blood pressure, reduction of renal blood flow, etc.), the target is moved in operation 640 and a new stimulation procedure is commenced. If a physiological of response does occur, then the target region is ablated in operation 650, and a physiological response is verified in operation 660. If no physiological response occurs (e.g., decrease of blood pressure, increase of renal blood flow, etc.), the ablation can continue. If a physiological of response does occur, then the target region is stimulated in operation 670, and a physiological response is measured in operation 680. If no physiological response occurs (e.g., increase of blood pressure, reduction of renal blood flow, etc. not exceeding a predetermined threshold), then the nerve in the target region is determined to be sufficiently ablated, and the procedure 600 can end in operation 690, optionally leading into another procedure (e.g., moving to, stimulating, and/or ablating another target region). If a physiological of response does occur (e.g., exceeding a predetermined threshold), then the nerve in the target region can be determined to be insufficiently ablated and further ablation in operation 650 can commence or be resumed. According to some embodiments, the predetermined threshold for evaluating a physiological response can be based, at least in part, on measurements taken in operation 630 after a first stimulation in operation 620. For example, a measurement taken in operation 630 may form the threshold by which a physiological response in operation 680 is evaluated, determine whether the physiological response after the stimulation in operation 670 does not match (e.g., does not exceed) a magnitude of the physiological response from operation 630. By further example, an ablation procedure can be determined to be successful if similar stimulation procedures before and after the ablation provide sufficiently different physiological responses.

According to some embodiments, the primary outcome measures are blood pressure and norepinephrine levels. Nerve area and immunohistochemical markers can be secondary outcome measures. To test for differences in blood pressure and serum norepinephrine pre- and post-treatment between the different experimental groups a repeated-measures ANOVA followed by a Tukey's post hoc test can be utilized. To determine if there are differences in kidney norepinephrine and nerve area between the different experimental groups an ANOVA can be performed followed by a Tukey's post hoc test. Statistical significance can be set at p<0.05.

According to some embodiments, systems and methods for imaging tissue using magnetic resonance imaging (“MRI”) techniques may be used. Thermal surgery guided by MRI systems and procedures can be used to selectively destroy tissue in a patient with localized heating, without adversely affecting tissue that is to remain substantially unaffected by the procedure. According to some embodiments, an MRI device 150 with RF coils can be designed to receive signals from tissues such as muscle, glandular tissue, and fat (among other tissues). An MRI pulse sequence is provided to obtain images that measure temperature in the tissues.

According to some embodiments, an MRI device 150 can include one or more radiofrequency (“RF”) coils and/or RF coil arrays embedded within a support portion 160, the treatment portion 170, and/or a modular portion 180. Radiofrequency coils can be utilized during an MRI procedure to monitor the activity and effect of a HIFU device 50. Results observed via an MRI procedure may be reported or transmitted to guide, initiate, or cease a HIFU therapy. For example, a control system governing positioning and orientation of a HIFU device 50 can be guided based on operation of an MRI device 150.

MRI systems may be used for planning surgery and/or during actual destruction of tissue. MRI systems using separate scanning sequences provide thermal level information and, in addition, also provide tissue information. Thus, the actual thermal level of the tissue can be ascertained using magnetic resonance imaging methods, and the ablation of the tissue can be observed using the MRI system.

According to some embodiments, an MRI device 150 for guiding HIFU operation comprises at least one of a coil that generates a static magnetic field, a RF coil, an x-gradient coil, a y-gradient coil, and a z-gradient coil. One or more coils allow sequences of currents to acquire PRF measurements and sequences to acquire T1 weighted images. There are several MRI methods may be used for measuring thermal levels using well-known MRI parameters, such as the spin-lattice relaxation time (“T1”). Sequence parameters—such as the time to repeat (“TR”), the time to echo (“TE”), and the flip angle—may be chosen by the user. For example, thermal level maps can be generated based on such procedures that provide T1 derived images evaluated with fast spoiled gradient echo sequences applied during the actual thermal therapy exposure. The parameters used are to some degree based on the tissue type and the precise evaluation of the behavior due to physiological or metabolic changes in the tissue during thermal therapy exposure. For example, TE, TR, and the flip angle of the spoiled gradient echo may be specified in the sequence.

The heated region may be imaged with the use of the MRI systems, employing a thermal level sensitive MR pulse sequence to acquire a thermal level “map” that is used basically to assure that the heat is being applied to the tissue and not to the surrounding healthy tissue. This is done by applying a quantity of heat that is insufficient to cause necrosis but is sufficient to raise the thermal level of the heated tissue. The MRI system thermal level map shows whether or not the heat is applied to the previously located tissue. The imaging system is also used in a separate scan sequence to create an image of the tissue intended to be destroyed. Using the imaging system in the prior art, the operator of the apparatus adjusts the placement of the radiation on the site of the tissue to be destroyed. The MR image of the tissue acquired in the separate scan determines in real time if necrosis is occurring and effectively ablating the tissue. However, the monitoring and guiding are provided using separate two-dimensional scan sequences.

Various methods for acquiring electromagnetic signals are known, in particular in the magnetic resonance imaging (MRI) field. They generally include subjecting the body to a high-intensity magnetic induction B0, typically between 0.1 and 3 Tesla. The effect of this induction is to orient the magnetic moments of the protons of the hydrogen contained in the water molecules of the body in a direction close to the main direction of the magnetic induction B0. The body part imaged is then subjected to a radiofrequency wave applied perpendicular to the magnetic induction B0 and the frequency of which is typically adjusted to the Larmor precession frequency of the hydrogen nucleus in the magnetic induction B0 in question. Immediately after the transmission of this radio frequency wave, the magnetic moments that have been subjected to the wave begin to oscillate around their equilibrium position and again take up a position along their original direction, close to that of the magnetic induction B0. During the relaxation, each water proton that has come into resonance creates, as a result, a relatively weak electromagnetic signal, called a magnetic resonance signal. This signal can then be detected by means of an appropriate detection module. Gradients of the magnetic induction B0 can be used in various spatial directions, so as to have different induction values between two points in space, each corresponding to an elementary volume of the body in question. The use of magnetic induction B0 gradients therefore allows spatial localization of the signal. The step of coding the space by means of the gradients is carried out between the proton excitation and the magnetic resonance signal reception.

In some exemplary methods, referred to as “time of flight” methods, the radio frequency waves are transmitted repeatedly and regularly, in a train of pulses. In some exemplary methods, referred to as “phase contrast” methods, takes advantage of the relationship that exists between the phase of the detected magnetic resonance signal and the rate of proton displacement in the body in question, to allow detection of blood vessels within the body. In some exemplary methods, a contrast product is injected into a body to enhance an image.

Various MRI methods may be used for measuring thermal levels using well-known MRI parameters, such as the spin-lattice relaxation time (“T1”). Sequence parameters—such as the time to repeat (“TR”), the time to echo (“TE”), and the flip angle—may be chosen by the user. For example, thermal level maps can be generated based on such procedures that provide T1 derived images evaluated with fast spoiled gradient echo sequences applied during the actual thermal therapy exposure. The parameters used are to some degree based on the tissue type and the precise evaluation of the behavior due to physiological or metabolic changes in the tissue during thermal therapy exposure. For example, TE, TR, and the flip angle of the spoiled gradient echo may be specified in the sequence. Sequence parameters may be used to localize the low-thermal level elevation induced by a focused ultrasound beam during both the planning and treatment.

According to some embodiments, magnetic resonance (MR) thermometry can be based on proton resonance frequency (PRF) shift to monitor temperature changes in an area heated by HIFU in MRI-guided HIFU equipment, further based on the phenomenon of the resonance frequency of the protons in water being offset (shifted) dependent on the temperature change. MR thermometry based on PRF-shift requires that a base image (MR phase image) before heating, also referred to as a reference image, be generated, with the reference image providing information on a reference phase. By subtraction from the phase image (also referred to as a heated image) acquired during heating or after heating, the exact value of the elevated temperature in the heated area can be determined.

As used herein, “thermal level” includes absolute temperature, relative temperature, temperature change, heat, change in heat, relative heat, thermal dosage, and other metrics related to thermal conditions.

Incorporated herein by reference are the following US patents and/or publications containing further teachings regarding MR imaging: US Pub. No. 2009/0275821, published on May 5, 2008; US Pub. No. 2006/0058642, published on Mar. 16, 2006; US Pub. No. 2010/0217114, published on Aug. 26, 2010.

According to some embodiments, application of focused sound energy to points around an artery has the result that sympathetic nerves are damaged and the artery is not damaged. The temperature of the artery may be substantially maintained by blood flow through the artery during the procedure while temperature of at least one nerve is elevated. Tissue near the nerves and the artery may be monitored by MRI or other means, whereby delivery of heat may be ceased when a thermal level exceeds a threshold.

According to some embodiments, a cooling catheter may be provided within the artery in a vicinity of the focal region of the focused sound energy. The cooling catheter provides maintenance of reduction of thermal levels in or around the artery to reduce or eliminate damage to the artery. According to some embodiments, a catheter may be provided at, along, or aligned with a target location within an artery. The catheter may provide a localizing signal to an MRI scanner or other device to identify the target location. The target location may identify where a focal region of the focused sound energy should be applied.

According to some embodiments, the method includes, as a result of the heating, lowering a blood pressure in a mammal. According to some embodiments, devices and methods disclosed herein may be used to treat Congestive Heart Failure (“CHF”) or related conditions, including hypertension. In addition to their role in the progression of CHF, the kidneys play a significant role in the progression of Chronic Renal Failure (“CRF”), End-Stage Renal Disease (“ESRD”), hypertension (pathologically high blood pressure) and other cardio-renal diseases. The functions of the kidneys can be summarized under three broad categories: filtering blood and excreting waste products generated by the body's metabolism; regulating salt, water, electrolyte, and acid-base balance; and secreting hormones to maintain vital organ blood flow. Without properly functioning kidneys, a patient will suffer water retention, reduced urine flow and an accumulation of waste toxins in the blood and body. These conditions result from reduced renal function or renal failure (kidney failure) and are believed to increase the workload of the heart. In a CHF patient, renal failure will cause the heart to deteriorate further as fluids are retained and blood toxins accumulate due to the poorly functioning kidneys.

It has been established in animal models that the heart failure condition results in abnormally high sympathetic activation of the kidneys. An increase in renal sympathetic nerve activity leads to decreased removal of water and sodium from the body, as well as increased renin secretion. Increased renin secretion leads to vasoconstriction of blood vessels supplying the kidneys, which causes decreased renal blood flow. Reduction of sympathetic renal nerve activity, e.g., via renal nerve ablation, may reverse or ameliorate processes.

According to embodiments, heating may be ceased for a period of time between any ablation procedure and a subsequent procedure on ipsilateral renal nerves. For example, a time period may be sufficient to allow inflammation to recede, scar tissue to begin forming, blood pressure to equilibrate, and any compensatory hypertensive effect from the contralateral kidney to manifest. For example, the time period may be greater or less than 1 day, 10 days, 100 days, and 1000 days. By further example, the time period may be equal to or greater than 1, 2, 3, 4, 5, 6, 7, 10, 15, 30, 60, 90, 120, or 180 days. By further example, the time period may be equal to or greater than 1, 2, 3, 4, 5, 6, 7, 8, 9, 10, 11, 12, 18, or 24 months.

According to some embodiments, devices and methods disclosed herein may be used in conjunction or combination with other devices and methods for achieving renal neuromodulation, including localized drug delivery (such as by a drug pump or infusion catheter), stimulation electric field, and laser therapy, inter alia.

Incorporated herein by reference are the following US patents and/or publications containing teachings regarding renal nerve ablation techniques: US Pub. No. 2010/0057150, published on Mar. 4, 2010; US Pub. No. 2010/0222854, published on Sep. 2, 2010; US Pub. No. 2008/0213331, published on Sep. 4, 2008.

### Examples

Initial catheter-based renal sympathetic denervation (RSD) studies demonstrated promising results in showing a significant reduction of blood pressure, while recent data were less successful. As an alternative approach, an objective of this example was to evaluate the feasibility of using magnetic resonance guided high intensity focused ultrasound (MRgHIFU) to perform RSD in a porcine model.

An intravascular fiber optic temperature probe was used to confirm energy delivery during MRgHIFU. This technique was evaluated both in a vascular phantom and in a normotensive pig model. Five animals underwent unilateral RSD using MRgHIFU and both safety and efficacy were assessed. MRI was used to evaluate the acoustic window, target sonications, monitor the near-field treatment region using MR thermometry imaging, and assess the status of tissues post-procedure. An intravascular fiber optic temperature probe verified energy delivery. Animals were sacrificed 6 to 9 days post-treatment and pathological analysis was performed. The norepinephrine present in the kidney medulla was assessed post-mortem.

All animals tolerated the procedure well with no observed complications. The fiber optic temperature probe placed in the target renal artery confirmed energy delivery during MRgHIFU, measuring larger temperature rises when the MRgHIFU beam location was focused closer to the tip of the probe. Following ablation a significant reduction (p=0.04) of cross-sectional area of nerve bundles between the treated and untreated renal arteries was observed in all of the animals with treated nerves presenting increased cellular infiltrate and fibrosis. A reduction of norepinephrine (p=0.14) in the kidney medulla tissue was also observed. There was no indication of tissue damage in arterial walls.

Performing renal denervation non-invasively with MRgHIFU was shown to be both safe and effective as determined by norepinephrine levels in a porcine model. This approach may be a promising alternative to catheter-based strategies.

Arterial hypertension represents a critical health challenge for millions of people, producing a well-established multiplication of risk for an array of cardiovascular diseases affecting 74.5 million adults in the United States. Appropriate adjustment of blood pressure is frequently challenging, despite the numerous pharmacologic options available. Indeed, roughly 40% of patients undergoing treatment have uncontrolled hypertension. A portion of this population has treatment resistant hypertension (TRH), which is identified in a patient when a therapeutic strategy of a diuretic and two other antihypertensive drugs fail to lower blood pressure values below 140/90 mmHg. While the prevalence of treatment resistant hypertension (TRH) in the uncontrolled hypertension population varies significantly in the literature, an approximate prevalence of 10-20%. Recognition of this common clinical problem has stimulated research exploring adjunctive non-pharmacological approaches. The well-characterized role of the sympathetic renal nervous system in initiating and maintaining hypertension has led to the development of technologies that target and interrupt sympathetic renal nerves residing in the arterial wall and perivascular soft tissue.

Numerous pre-clinical and clinical trials have investigated endovascular catheter-based technologies as a primary or adjuvant treatment for TRH. Initial clinical studies reported promising results by significantly lowering both systolic and diastolic blood pressure (6,7), even after 3 years of follow-up. Those studies resulted in an increased interest in the technique and usage at multiple worldwide sites. However, a randomized, multicenter clinical trial applying catheter-based RSD in humans did not show a significant decrease in blood pressure when compared to the sham-control group. Conversely, a prospective, open-label randomized control trial demonstrated that in subjects treated with RSD in addition to a standardized stepped-care antihypertensive treatment (SSHAT) had reduced amubulatory blood pressure more than SSHAT alone.

Even though the catheter-based technologies have shown variable results, the procedure has demonstrated significant promise justifying the investigation of both catheter-based and other RSD treatment options.

High intensity focused ultrasound (HIFU) is an established treatment option in various disorders and has been proposed as an alternative energy delivery source for RSD therapy. Recently both an ultrasound- and MRI-guided approach demonstrated feasibility using HIFU to perform RSD in normotensive canine and porcine models with mixed efficacy results. This example furthers those feasibility assessments through performing renal denervation using MRgHIFU in a normotensive porcine model.

Methods: In MRgHIFU therapy, MRI is used in all aspects of the treatment process including planning, real-time procedure monitoring and assessment. Ideally, real-time MR thermometry is used to measure the temperature elevation during the procedure and predict the tissue damage based on the accumulated thermal dose. However, imaging artifacts due to the presence of motion (including arterial, respiratory and peristalsis motion) and the presence of fat render standard proton resonance frequency thermometry techniques inaccurate. Because of these effects, obtaining accurate MR thermometry measurements in the area immediately surrounding the renal artery (i.e. regions extending approximately 1 cm away radially from the artery centerline) is extremely challenging. In this work, real-time MR thermometry measurements were not obtained in the regions immediately surrounding the renal artery during the RSD procedure. Therefore, in order to obtain a real-time assessment of the energy delivered to the target area surrounding the renal artery by the HIFU beam, an intravascular fiber optic temperature probe was placed in the targeted artery and continuously monitored during the RSD procedure. The use of this invasive temperature probe was evaluated in a vascular phantom as well as an in vivo normotensive porcine model.

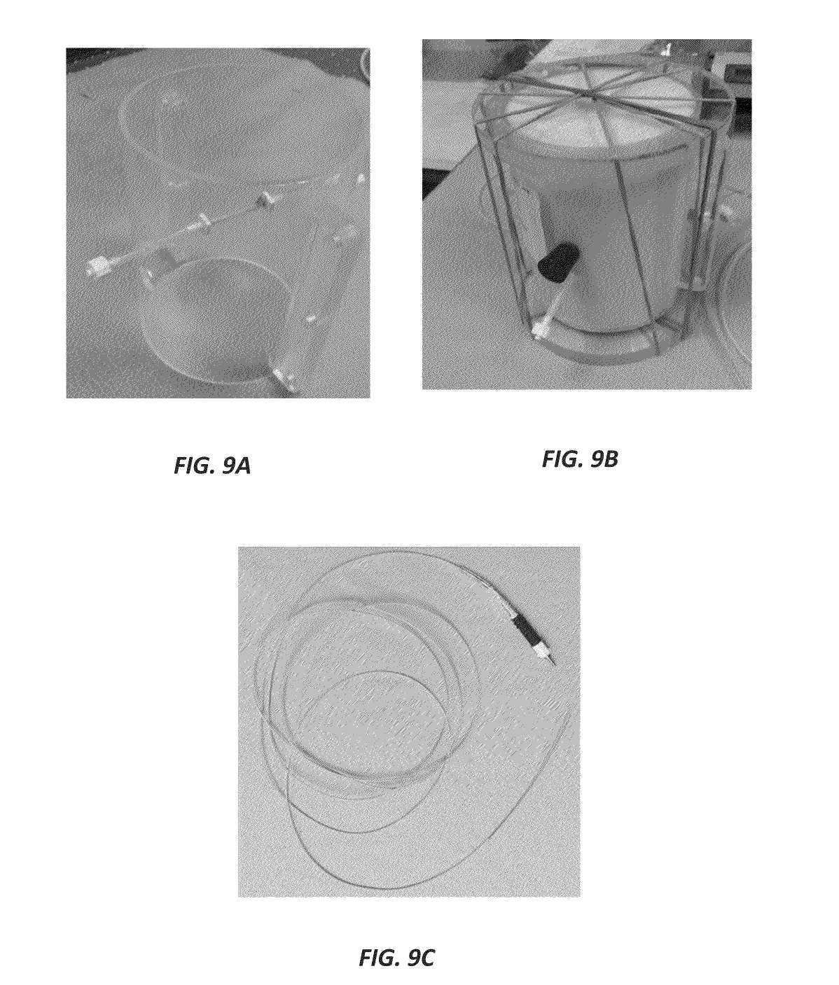

Vascular phantom preparation: In order to validate the use of an intravascular temperature probe, a vascular phantom was developed. FIGS. 9A-C show an excised rabbit aorta secured in an acrylic phantom mold. A fiber optic temperature probe (Neoptix, Quebec, Canada) was placed in the vessel such that fluid could flow around the probe through the vessel and tissue-mimicking gelatin was poured around the vessel. The phantom was mounted on a pre-clinical MRgHIFU system (256-element phased-array transducer, f=1 MHz, 2×2×8 mm focal spot size, Image Guided Therapy, Inc., Pessac, France) and the entire assembly was placed in a Siemens Trio 3 Tesla MRI scanner (Erlangen, Germany). Degassed, deionized water was used to both acoustically couple the phantom to the transducer and to perfuse the embedded vessel.

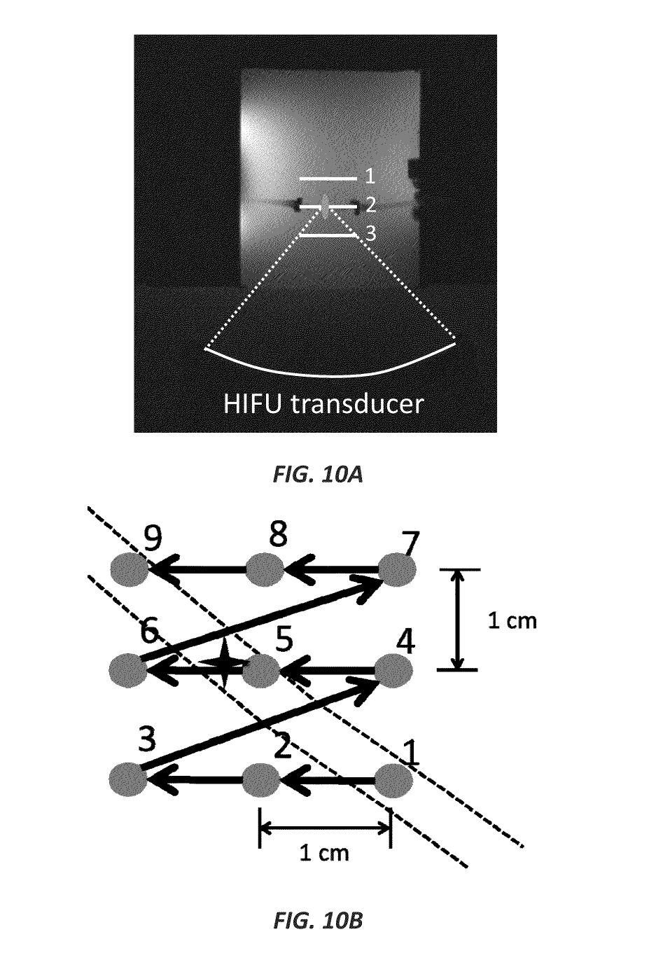

Multiple sonications were performed in a three plane, 27-point raster pattern centered on the embedded excised artery at two flow rates, 40 and 80 mL/min (FIGS. 10A-B). Each point was sonicated for 20 seconds at 35 W and 20 seconds of cooling time elapsed before the following point was sonicated. The fiber optic temperature probe recorded the temperature in the artery every 0.5 s. MR thermometry during the experiment was achieved with a 3D segmented-EPI gradient echo sequence (TR/TE=40/10 ms, flip angle=40°, 1.6×1.6×3 mm resolution, 112×256×24 mm FOV, ETL=9). Two, 2-channel surface RF coils were placed on the sides of the cylindrical phantom holder to provide sufficient SNR for the example.

The position of each focal spot was determined by the location of the peak temperature as measured by the MR temperature imaging (MRTI). The temperature rise (Tᵣᵢₛₑ=Tₚₑₐₖ−T_{baseline}) detected by the fiber optic probe at each sonication location was also determined.

Animal preparation: All applicable institutional and national guidelines for the care and use of animals were followed. Five normotensive female Yorkshire pigs (40-50 kg) were included in the example. Anesthesia was induced with a Telazol, Ketamine and Xylazine cocktail (4.4, 2.2 and 2.2 mg/kg, respectively) and maintained with isoflurane (1-3%, inhaled). Hair on the back of the animal was removed with clippers and a depilatory cream to improve acoustic window quality.

Similar to the vascular phantom, a fiber optic temperature probe was placed in the right renal artery through percutaneous access of the femoral artery under fluoroscopy guidance. The temperature probe was sheathed in a 6 French multipurpose angiographic catheter with the tip of the temperature probe extended approximately 1 cm distal to the end of the angiographic catheter.

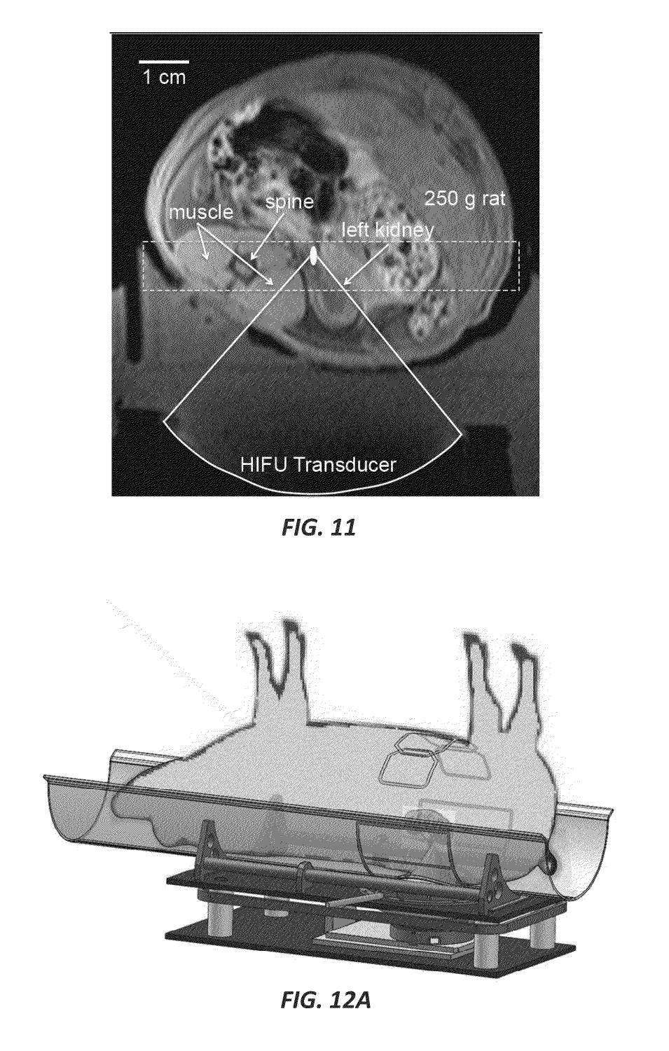

In a study, nerve stimulation was achieved using an MRgHIFU system. An RF coil phased array was used to obtain high SNR images during all phases of the procedure. An example of a T1w axial image of a 250 g rat placed on the MRgHIFU system is shown in FIG. 11. Both the left and right renal arteries can be accessible by the HIFU beam simultaneously or sequentially.

MRgHIFU renal sympathetic denervation procedure: RSD in the porcine model was performed using the same pre-clinical MRgHIFU system and MRI scanner as in the vascular phantom study. The animal was placed on top of the MRgHIFU system in a custom support holder in an oblique supine position with an integrated 9-channel RF receive coil surrounding the animal (seen schematically in FIG. 12A). MR imaging was used to accurately position the animal, evaluate the acoustic window and plan the sonication locations around the target renal artery (3D T1-weighted Volumetric Interpolated Breath-hold Examination [VIBE], T2-weighted Turbo Spin Echo [TSE]).

Because of the location of the bowel in all the animals treated in this example, RSD using MRgHIFU was performed in all animals unilaterally on the right side, with the left side serving as a control. Several single point sonications (as detailed in Table 1) were applied to the regions at a close anatomical proximity to the right renal artery.

TABLE 1
MRgHIFU sonication details for each of the treated animals.

| | No. of | Sonication | Acoustic | Total | |
|---|---|---|---|---|---|
| Animal | sonication | time/point | power | energy | Δtime |
| ID | points | (seconds) | (W) | (kJ) | (days) |
| 1 | 7 | 20 | 83 | 11.6 | 6 |
| 2 | 26 | 20 | 81 | 42.1 | 6 |
| 3 | 17 | 20 | 82 | 27.9 | 7 |
| 4 | 16 | 20 | 120 | 38.4 | 9 |
| 5 | 16 | 45 | 140 | 100.8 | 9 |

In general, the number of sonications applied per animal was a function of the overall length of the renal artery and the available study time. While the transducer power output was approximately 80 W for animals 1 through 3, the power was increased in animals 4 and 5. The animal's SpO₂, end tidal CO₂ and body temperature were monitored continuously throughout the MRgHIFU procedure.

Due to the significant susceptibility artifacts from peristalsis, blood flow artifacts and the presence of fat in the target region, temperature measurements in the area immediately surrounding the renal artery were not obtained in this example. MR thermometry techniques were however used to monitor the treatment in the near field of the ultrasound beam. The 3D imaging volume, as indicated in FIG. 12B, was placed such that any interference between the ultrasound beam and transverse process could be monitored using real-time MRTI (3D Segmented-Echo Planar Imaging [EPI]). The MRTI measurements were used to calculate the thermal dose, as defined by Sapareto and Dewey, deposited in the tissues during the course of the MRgHIFU RSD treatment. T2-weighted TSE and post-contrast VIBE scans (0.05 mmol/kg, MultiHance, Bracco Diagnostics Inc.) were used to evaluate surrounding tissues post procedure. Relevant MR parameters for all listed sequences are located in Table 2.

TABLE 2
Typical MRI parameters used in the in vivo experiments.

| Pulse | TR | TE | Flip Angle | Resolution | FOV |
|---|---|---|---|---|---|
| Sequence | (ms) | (ms) | (°) | (mm) | (mm) |
| 3D T1w | 4.33 | 1.97 | 9 | 1.2 × 1.7 × 3 | 380 × 286 × 168 |
| VIBE | | | | | |
| 2D T2w | 2000 | 89 | 180 | 1.3 × 1.4 × 4 | 320 × 280 × 72 |
| TSE | | | | | |
| 3D seg- | 35 | 11 | 25 | 2 × 2 × 3 | 256 × 192 × 30 |
| EPI | | | | | |
| MRTI | | | | | |

Tissue Processing: Six to nine days after the renal denervation procedure, the animal was sacrificed and a necropsy performed. Bilateral kidneys, renal arteries and surrounding tissue, abdominal aorta, and adjacent muscle were examined for any gross abnormalities. Tissue was fixed for 24 to 48 hours in 10% formalin. Each renal artery was divided into four equal segments with the segment closest to the aorta designated as region 1 and the segment closest to the kidney designated as region 4. The segments were dehydrated in increasing concentrations of alcohol, embedded in paraffin, and then sectioned (5 μm). One haematoxylin and eosin (H&E) slide was prepared and a section from each segment was analyzed.

Morphometric Analysis: The stained sections were digitally scanned with the ScanScope® XT system and visualized using ImageScope software in eSlideManager (Aperio/Leica Biosystems, Vista, Calif.). Each arterial segment (regions 1-4) was analyzed using positive pixel count and measurement tools of ImageScope software to determine nerve count, cross-sectional nerve and artery area, and distance from nerve to arterial lumen. For calculation and analysis of mean nerve area only nerves that were greater than 5,000 μm² and smaller than 70,000 μm² were included in the calculation.

Norepinephrine-ELISA: At necropsy both kidneys were placed in ice-cold phosphate buffered saline, segments of the medulla were isolated, weighed, homogenized in 0.8M EDTA, and then frozen (−80° C.). The levels of norepinephrine (ng/mL) in the homogenate were measured via enzyme-linked immunosorbent assay (ELISA) following the manufacturer's instructions (Rocky Mountain Diagnostics, Colorado Springs, Colo.).

Statistics: Nerve area and kidney norepinephrine (NE) levels were compared between the treated and non-treated sides with a paired t-test (JMP Pro 11; SAS; Cary, N.C.), with significance set at p<0.05.

MRgHIFU RSD procedure: A representative pre-RSD treatment acoustic window evaluation using T1-weighted (T1w) 3D VIBE images, which is utilized to evaluate effective transducer positioning and acoustic coupling of the transducer to the animal's skin, is shown in FIG. 12B. The spine, bowel, kidney, aorta and renal artery are all easily visualized without contrast agent allowing the animal to be positioned such that the interaction of the ultrasound beam with high acoustic impedance anatomy was minimized. The angiographic catheter housing the fiber optic temperature probe is seen in the aorta and at the renal artery junction.

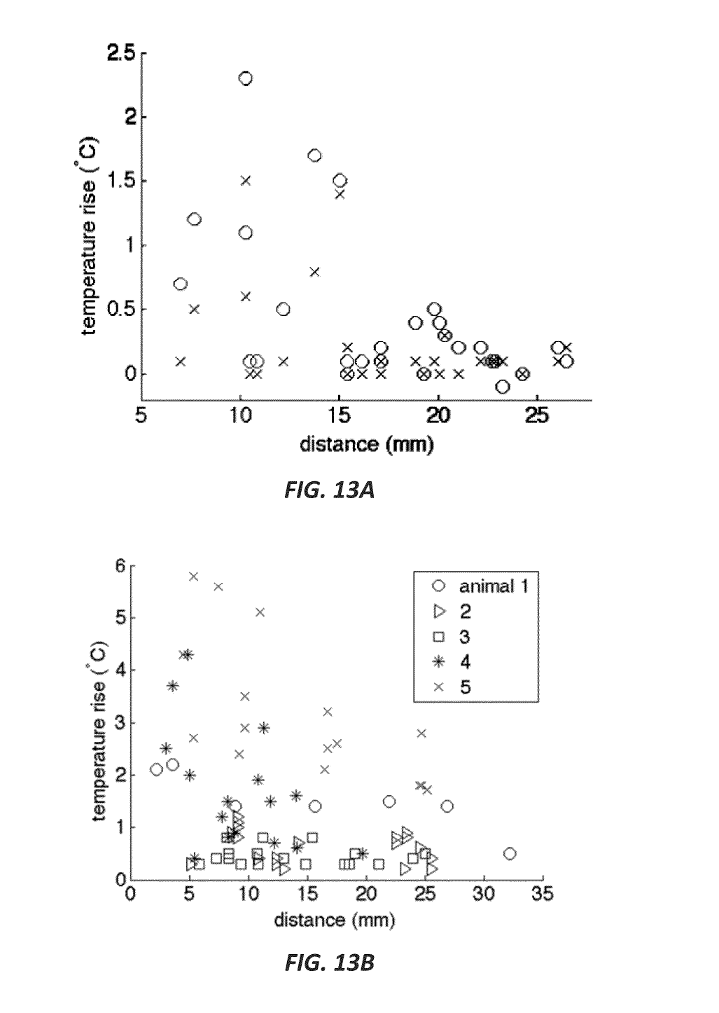

Results—Vascular phantom: The results shown in FIG. 13A from the vascular phantom experiments demonstrate that MRgHIFU sonications performed closer to the tip of the fiber optic temperature probe resulted in a higher measured temperature rise. This decreasing trend of temperature rise as a function of sonication distance from the probe tip to the focused ultrasound beam location is seen at both the 40 and 80 mL/min flow rate. Predictably, overall higher temperature rises were observed at the lower flow rate.

The fiber optic temperature probe placed in the renal artery on the treated side provided verification of energy delivery that was independent of MR measurements. The temperature rise measured by the probe as a function of distance to the targeted MRgHIFU beam location is shown in FIG. 13B. Similar to the observations made in the vascular phantom, the temperature rise measured by the fiber optic temperature probe decreases as the distance between the probe tip and the MRgHIFU beam location increases. While the magnitude of this relationship varies, as seen in Table 3, the trend is present for all evaluated animals.

TABLE 3
Procedure results for all treated animals. Slope is the
decreasing temperature trend as a function of distance
from fiber optic probe tip to MRgHIFU focus location.

| | Fiber optic temperature | | Near-field MRI measurements | |
|---|---|---|---|---|
| | probe | | Edema | Volume |
| Animal | Slope | R² | (y/n), volume | (mm³) ≧240 |
| ID | (° C./mm) | value | (mm³) | CEM 43° C. |
| 1 | −0.04 | 0.74 | Yes, 1185 | 125 |
| 2 | −0.007 | 0.020 | Yes, 328 | 607 |
| 3 | −0.004 | 0.016 | No | 25 |
| 4 | −0.13 | 0.27 | No | 1002 |
| 5 | −0.12 | 0.47 | Yes, 621 | 123 |

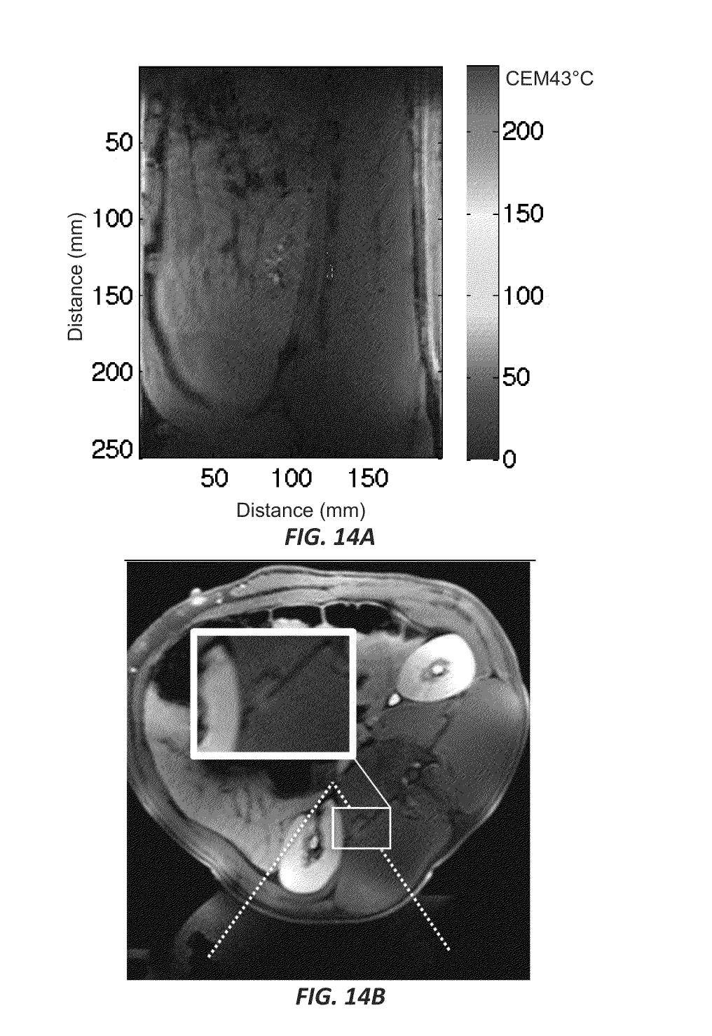

The real-time MRTI monitoring that was performed in the near-field of the MRgHIFU beam confirms that in all animals, some energy was deposited in the muscle area surrounding the transverse process. FIG. 14A shows the cumulative thermal dose deposited during an RSD procedure overlaid on a coronal magnitude image. The volume of tissue in the near field that received possible permanent damage (thermal dose>240 CEM43° C.) ranged from 25 to 1000 mm³ as listed in Table 3. This potential damage was confirmed by delayed contrast-enhanced T1w VIBE image (FIG. 14B). In 3 out of 5 of the animals, the presence of edema was detected by post-RSD treatment assessment. The existence of edema and the corresponding size of the enhancing regions is reported in Table 3.

MRgHIFU RSD procedure safety: All animals recovered quickly from the RSD procedure with no observed complications. During necropsy all anatomical structures between the energy source and the target region were carefully observed including the skin, muscle tissue, spine, renal arteries and veins, ureters, liver, bowels, and kidneys. Based on gross histological examination, there was no detectable tissue damage along the acoustic beam, other than in the target region. Importantly, injuries of the arterial wall were not observed.

Gross examination revealed several hemorrhagic spots located in the fatty tissue around the treated renal arteries. The length of the renal artery from the aorta to the bifurcation was not found to be significantly different (p=0.17) between the treated (3.4 cm±0.5 cm) and the control side (3.1 cm±0.2 cm). The distance from the nerves to the lumen (endothelium) of the renal artery was determined for both the treated and control sides (Table 4).

TABLE 4
Distance from the renal nerves to the endothelium of the renal artery
as a function of anatomical position for treated and untreated arteries. Each table cell represents
the number of nerves visible in a single slide prepared from the designated region with the
percentage of nerves for that given side. There is a proximal to distal distribution, while region 1
is closest to the aorta and region 4 closest to the kidney.

| Distance | | | | | | | | |
|---|---|---|---|---|---|---|---|---|
| from | | | | | | | | |
| lumen | TREATED ARTERIES | | | | CONTROL ARTERIES | | | |
| (mm) | Region 1 | Region 2 | Region 3 | Region 4 | Region 1 | Region 2 | Region 3 | Region 4 |
| 0-1 | 1 | — | 1 | 1 | — | 1 | 3 | 1 |
| | (0.9%) | | (0.9%) | (0.9%) | | (1.2%) | (3.5%) | (1.2%) |
| 1-2 | 5 | 13 | 26 | 38 | 4 | 7 | 24 | 16 |
| | (4.3%) | (11.1%) | (22.2%) | (32.5%) | (4.7%) | (8.2%) | (28.2%) | (18.8%) |
| 2-3 | 2 | 8 | 10 | 2 | 2 | 12 | 4 | 6 |
| | (1.7%) | (6.8%) | (8.6%) | (1.7%) | (2.4%) | (14.1%) | (4.7%) | (7.1%) |
| 3-4 | 1 | 2 | 4 | — | — | 1 | 1 | 1 |
| | (0.9%) | (1.7%) | (3.4%) | | | (1.2%) | (1.2%) | (1.2%) |
| 4-5 | — | — | — | — | — | — | 2 | — |
| | | | | | | | (2.4%) | |
| >5 | — | 3 | — | — | — | — | — | — |
| | | (2.6%) | | | | | | |

A total of 83 nerves on the treated side and 69 nerves on the control side (Table 4) met the inclusion criterion. Thirty-nine nerves that were smaller than 5 μm² on the treated side and 49 on the control side were excluded. There were 14 nerves on the treated side that exceeded 70 μm² and 12 on the control side. The majority of the nerves were located within 3 mm from the lumen of the artery (90% control and 96% treated). Regionally, a majority of nerves were located in regions 3 and 4, closer to the renal pelvis, both on the control (73%) and treated (71%) sides. There was also no significant difference in renal artery area between the treated side (6.03±1.53 mm²) and the control side (6.70±2.04 mm², p=0.27). There were no histological indications of damage to the renal artery as a result of the MRgHIFU RSD procedure.

MRgHIFU RSD procedure efficacy: Cumulative nerve area on the treated side was statistically smaller than the cumulative nerve area on the control side, with all of the animals treated with MRgHIFU having reduced nerve area on the treated side (Table 5, p=0.04).

TABLE 5
Ratio of Treated to Control Arteries
for Different Outcome Measures.

| Animal # | 1 | 2 | 3 | 4 | 5 |
|---|---|---|---|---|---|
| Energy delivered (kJ) | 21.6 | 42.1 | 27.9 | 38.4 | 100.8 |
| Nerve area ratio | 0.76 | 0.83 | 0.50 | 0.81 | 0.80 |
| Medulla norepinephrine ratio | 0.9 | 0.86 | 0.83 | 0.50 | 0.35 |

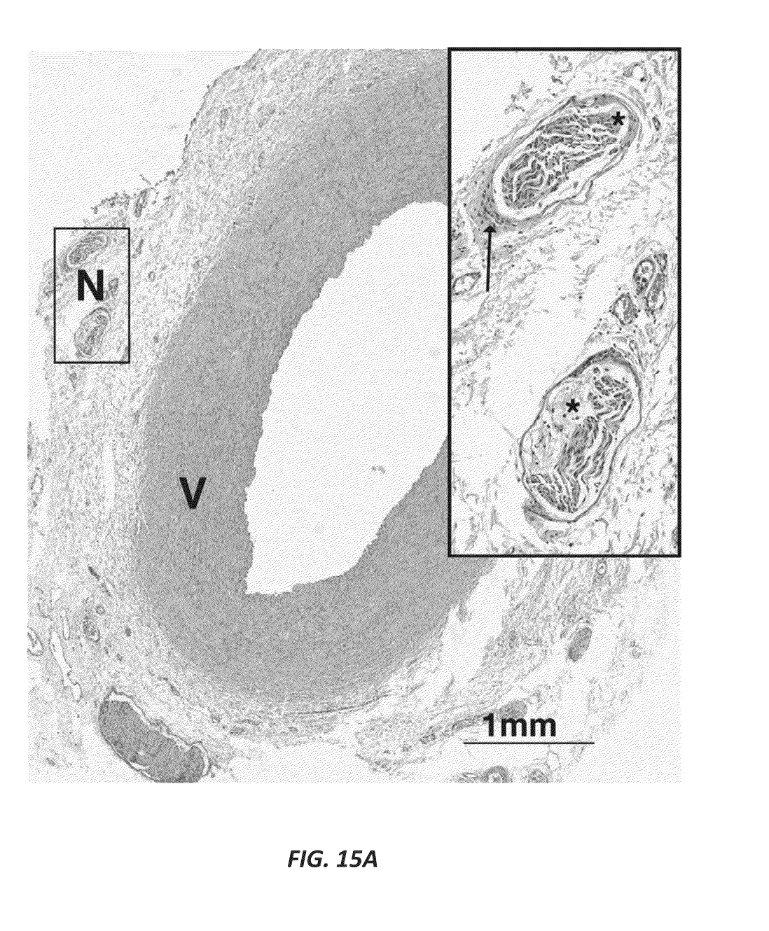

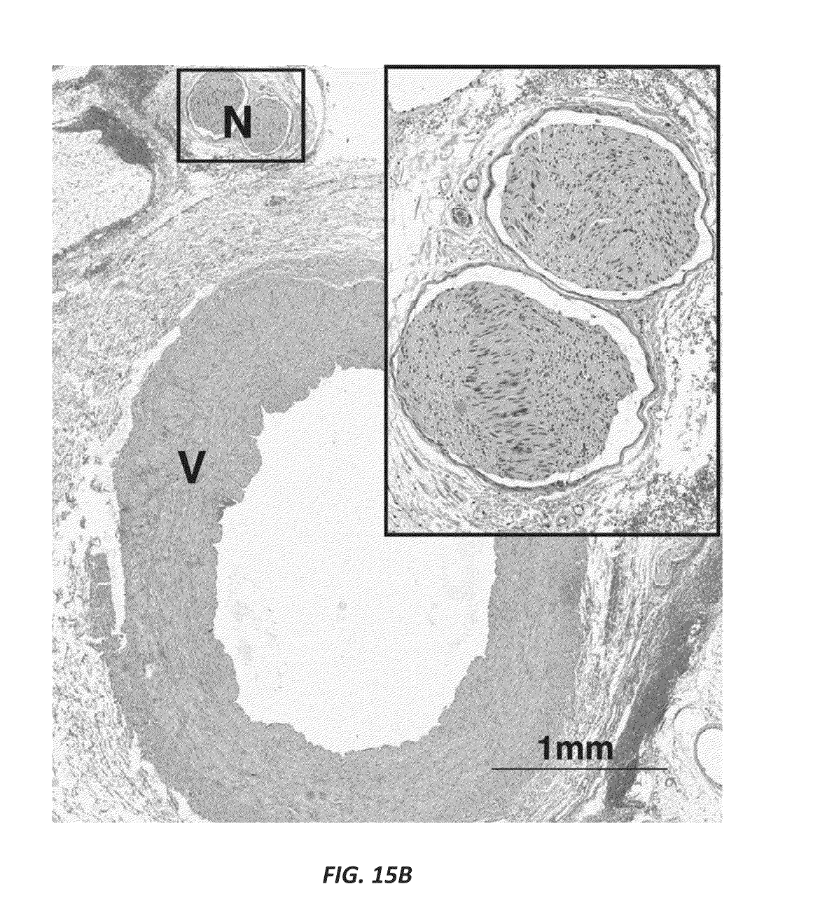

The mean nerve area on the treated side was roughly 25% smaller than the control side (Nerve Area_{treated/}Nerve Area_{control}=0.74±0.14, Table 5). FIGS. 15A-B shows the morphological changes observed, with the nerves on the treated side having increased cellular infiltrate, fibrosis, and shrunken appearance all of which indicate damage to the nerve. The ratio of norepinephrine in the treated and control kidneys decreased following renal ablation in all 5 of the animals evaluated (Table 5), though this decrease was not found to be statistically significant between the treated and non-treated side (p=0.14). The absolute values for norepinephrine ranged from approximately 500-1800 on the treated side and 1000-3300 on the control side as shown in Table 5.

MRgHIFU RSD Efficacy: This example has demonstrated the feasibility of using MRgHIFU to perform RSD in a normotensive porcine model safely, resulting in nerve bundle damage. The norepinephrine ratio measured directly from the kidney medulla tissue was reduced post-RSD procedure when comparing the treated with contralateral control kidney indicating successful RSD was performed. While the number of animals treated in this feasibility study was small, this measured reduction increased with applied energy indicating a potential dose effect that should be explored further in future studies. This preliminary finding agrees with RSD procedures performed with catheter methods. In the Simplicity HTN-3 trial, there was a positive correlation between the number of ablation attempts and the decrease of blood pressure. The reduction seen in the norepinephrine data is supported by the histological appearance of damaged renal nerves. In addition the cross-sectional area of the nerve was reduced on the treated side. This result is similar to other studies that have shown that nerve atrophy is a common indication of nerve damage, as observed following renal ablation and other common nerve injures and nerve injury models.

While the difficulties of obtaining accurate MR thermometry data at the treatment area prevented acute assessment of the success of the MRgHIFU procedure, the independent temperature measurements assessed with the intravascular fiber optic temperature probe provided confirmation of energy delivery. While the temperature rise measured by the probe for each sonication point did exhibit both inter- and intra-animal variability, in general higher temperature rises were measured when the MRgHIFU beam focus was located close to the probe tip. Obviously one of the main advantages of performing RSD with MRgHIFU is that the procedure would be completely non-invasive. Therefore, while using an intravascular fiber optic probe when performing RSD with MRgHIFU is not a desired aspect of future clinical work, this example has demonstrated that it can provide valuable information and qualitative treatment confirmation in pre-clinical studies. Therefore, while MR thermometry was not able to predict an acute treatment assessment, the use of the temperature probe did demonstrate the MRgHIFU beam was focused in close proximity to the renal artery. This result extends the assessment that has been performed in other HIFU RSD studies.

This example did not compare blood pressure measurements before and after the RSD procedure. Similar to other groups, we found separating the effect of the RSD procedure and anesthesia on blood pressure to be quite difficult. Indeed, whether RSD affects blood pressure in normotensive animals remains a matter of debate. For these reasons kidney medulla norepinephrine concentration is reported as the primary efficacy outcome for this example, a proven robust marker for effective renal nerve destruction. The norephinephrine reduction ranging from 10 to 65% post-RSD MRgHIFU procedure compares to other clinical studies where analysis from 10 patients revealed a mean reduction in norepinephrine spillover of 47% at 1 month after bilateral RSD. These numbers also compare to other pre-clinical RSD study performed with HIFU studies. In one study, a 51% reduction in plasma norephinephrine was observed 6 days post procedure. Conversely, in another study, no significant change in was observed in the renal parenchyma norepinephrine concentration.

MRgHIFU RSD Safety: While edema around the transverse process was observed in three animals, no tissue effect was observed during necropsy. Although the majority of the entire kidney is in the near field of the ultrasound beam, as seen in FIG. 13A, there was no observable damage to the organ. In addition, since the focal spot of the transducer is cigar shaped approximately 2×2×8 mm in size, it is likely that the MRgHIFU beam focus may have directly targeted the renal artery. Despite this possibility, there was no indication of renal artery wall damage in any of the analyzed histological sections.

The real time monitoring of the near-field regions during the MRgHIFU RSD treatment may potentially increase the safety of the overall procedure. Other studies have documented the potential of near-field heating buildup, particularly in cases where multiple sonications are executed from a fixed acoustic window, as was the case in this example.

Model Applicability: A porcine model was selected for this example due to similarities of the porcine cardiovascular system to human anatomy. In this example, the highest nerve bundle density is at the distal part of the renal artery, close to the kidney hilum. However, others have also reported the opposite with more nerve fibers closer to the aorta. This variability of results indicates that when conducting an ablation procedure it will likely be more effective if a greater region of the nerves around the artery is ablated to account for inter-patient variability.

Other anatomical features including the bowel and spinal column vary quite substantially between humans and porcine. The vertebrae of the porcine spinal column exhibits prominent transverse process causing aberration of the acoustic beam as assessed by the edema presence post-RSD procedure. Conversely, in humans the distance of the bowel to the left renal artery is not as close as in pigs. This difference would allow for bilateral renal artery ablation in humans. Indeed, human trials with ultrasound-guided HIFU are ongoing (clinicaltrials.gov, NCT02029885).

While the goal of RSD is to destroy the renal artery nerves with a negligible amount of collateral damage, it is difficult to determine the damage mechanism in this example. In this example the total delivered energy per animal varied from 10-100 kJ. Other RSD HIFU studies reported total energy delivery of 18 kJ and a mean of 26.2 kJ per animal with varied efficacy results. This variability indicates that successful treatment outcome is a function of applied dose as well as animal position and size.

Limitations: Normotensive animals were used in this example and were treated unilaterally, which likely limits the efficacy results observed. Due to the location of the bowel, only the right side could be treated introducing a potential bias in the example. No conclusions can be made regarding the long-term effects of RSD performed with MRgHIFU since the longest time span from ablation to renal nerve and kidney tissue analysis was nine days. We are currently exploring this question in ongoing pre-clinical studies. In addition, it should be noted when norepinephrine levels are assessed directly from the kidney tissues as done in this example, it does not allow the comparison of norepinephrine levels pre-RSD MRgHIFU procedure. There is the possibility that the reduction of norepinephrine may be due to other physiological changes including a change in stress level or vasoconstriction. However, in spite of these potentially confounding factors, the encouraging reduction in norepinephrine in the kidney medulla between the treated and control sides indicated that there was a dose ranging effect, which provides useful information to guide future study design.

MRgHIFU is a completely non-invasive technology that has the potential of being a valid RSD procedure technique. While arterial damage during catheter-based techniques has been rare, MRgHIFU would have no impact on vascular structure. It would also overcome any issues with renal artery anatomy. In addition, performing the procedure under MR guidance can allow for detailed treatment planning monitoring as well as a non-contrast angiographic method.

This example demonstrates feasibility of performing RSD using MRgHIFU in a porcine model. Soft-tissue contrast achieved by MR guidance is advantageous in pre-procedural planning, ensures accurate targeting and allows for exact visualization of the region of interest. While MR thermometry provided real-time monitoring of critical adjacent structures in the near-field during the procedure, an intravascular fiber optic temperature probe provided real-time feedback at the target area. MRgHIFU has the potential to be a valid technique for non-invasively performing RSD. Future studies will evaluate this approach in a hypertensive animal model with a longer follow-up and efforts will be made to improve MR thermometry techniques around the renal arteries.

### Systems

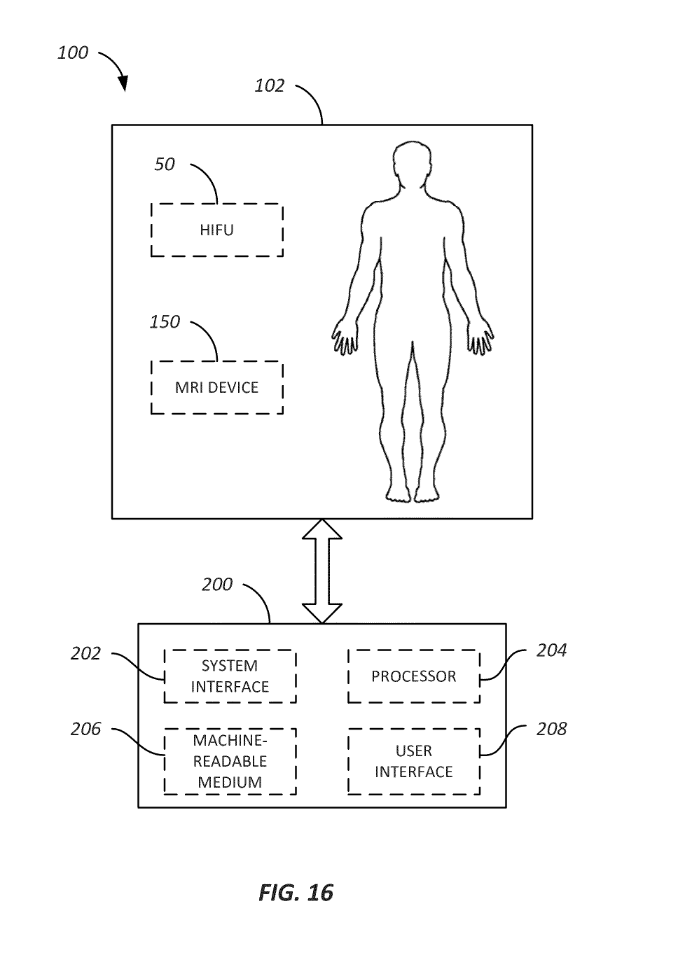

According to some embodiments, as shown in FIG. 16, an ablation system 100 may comprise an operation system 102 and a control system 200. Operation system 102 may comprise a HIFU device 50 and an MRI device 150 for performing operations on a patient. According to some embodiments, a control system 200 is provided to control, monitor, or interact with one or more components of operation system 102, such as HIFU device 50 and MRI device 150.

According to some embodiments, as shown in FIG. 16, a control system 200 may comprise a system interface 202, a processor 204, a machine-readable medium 206, a user interface 208, and other components as appropriate to produce the desired functionalities of the control system 200.

The control system 200 may include a processor 204 for executing instructions and may further include a machine-readable medium 206, such as a volatile or non-volatile memory, for storing data and/or instructions for software programs. The instructions, which may be stored in a machine-readable medium 206, may be executed by the control system 200 to control and manage access to the various networks, as well as provide other communication and processing functions. The instructions may also include instructions executed by the control system 200 for various user interface devices, such as a display and a keypad. The control system 200 may include an input port and an output port. Each of the input port and the output port may include one or more ports. The input port and the output port may be the same port (e.g., a bi-directional port) or may be different ports.

The system 200 can include, in addition to hardware, code that creates an execution environment for the computer program in question, e.g., code that constitutes processor firmware, a protocol stack, a database management system, an operating system, or a combination of one or more of them stored in an included memory 204, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, and/or any other suitable storage device, for storing information and instructions to be executed by the processor 204. The processor 204 and the medium 206 can be supplemented by, or incorporated in, special purpose logic circuitry.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

As used herein, a “processor” can include one or more processors, and a “module” can include one or more modules.

In an aspect of the subject technology, a machine-readable medium is a computer-readable medium encoded or stored with instructions and is a computing element, which defines structural and functional relationships between the instructions and the rest of the system, which permit the instructions' functionality to be realized. Instructions may be executable, for example, by a system or by a processor of the system. Instructions can be, for example, a computer program including code. A machine-readable medium may comprise one or more media.

The control system 200 may be implemented using software, hardware, or a combination of both. By way of example, the control system 200 may be implemented with one or more processors. A processor may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device that can perform calculations or other manipulations of information.

A machine-readable medium can be one or more machine-readable media. Software shall be construed broadly to mean instructions, data, or any combination thereof, whether referred to as software, firmware, middleware, microcode, hardware description language, or otherwise. Instructions may include code (e.g., in source code format, binary code format, executable code format, or any other suitable format of code).

Machine-readable media may include storage integrated into a processing system, such as might be the case with an ASIC. Machine-readable media may also include storage external to a processing system, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device. Those skilled in the art will recognize how best to implement the described functionality for the control system 200. According to one aspect of the disclosure, a machine-readable medium is a computer-readable medium encoded or stored with instructions and is a computing element, which defines structural and functional interrelationships between the instructions and the rest of the system, which permit the instructions' functionality to be realized. In one aspect, a machine-readable medium is a non-transitory machine-readable medium, a machine-readable storage medium, or a non-transitory machine-readable storage medium. In one aspect, a computer-readable medium is a non-transitory computer-readable medium, a computer-readable storage medium, or a non-transitory computer-readable storage medium. Instructions may be executable, for example, by a client device or server or by a processing system of a client device or server. Instructions can be, for example, a computer program including code.

An interface (e.g., 202 and/or 208) may be any type of interface and may reside between any of the components shown in FIG. 16. An interface may also be, for example, an interface to the outside world (e.g., an Internet network interface). A transceiver block may represent one or more transceivers, and each transceiver may include a receiver and a transmitter. A functionality implemented in a control system 200 may be implemented in a portion of a receiver, a portion of a transmitter, a portion of a machine-readable medium, a portion of a display, a portion of a keypad, or a portion of an interface, and vice versa.

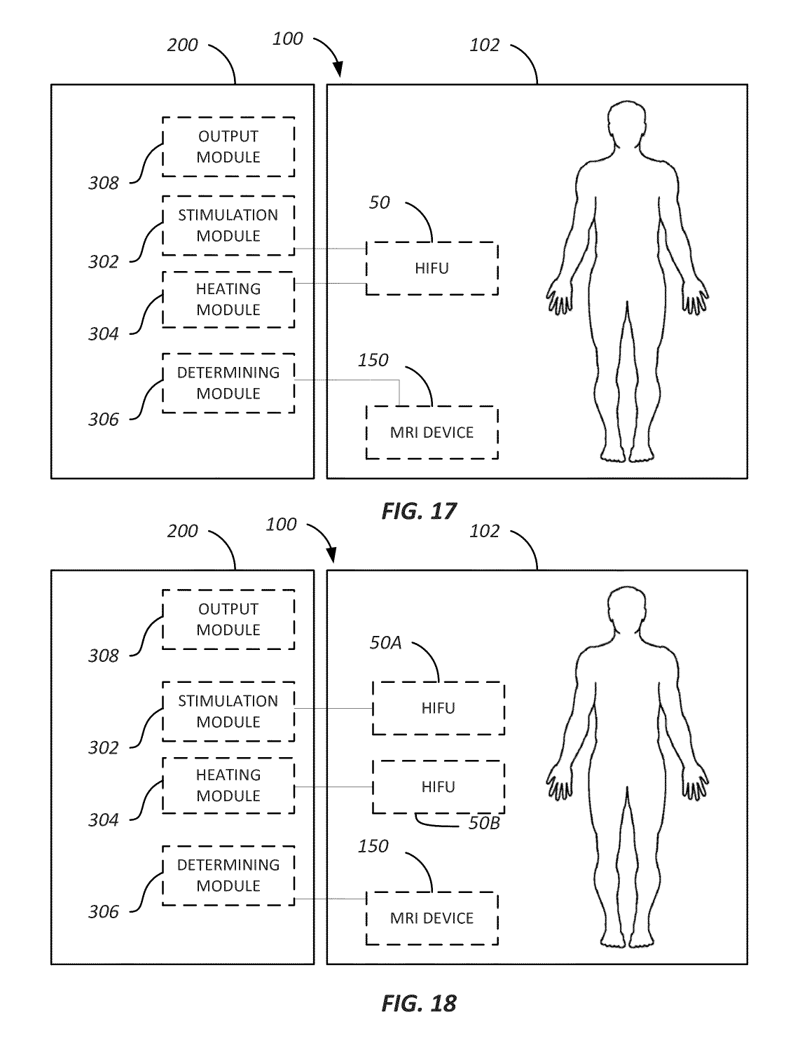

According to one or more embodiments, as shown in FIGS. 17 and 18, a control system 200 can include a stimulation module 302, a heating module 304, a determining module 306, and/or an output module 308. The heating module 304 can be configured to heat a region with ultrasound energy from an ultrasound device 50. The stimulation module 302 can be configured to stimulate a region with ultrasound energy from the ultrasound device 50. According to one or more embodiments, as shown in FIG. 17, the stimulation module 302 and the heating module 304 can be connected to and/or effectuate operation of the same ultrasound device 50. According to one or more embodiments, as shown in FIG. 18, the stimulation module 302 and the heating module 304 can be connected to and/or effectuate operation of different ultrasound devices 50A,B, respectively. The determining module 306 can be configured to determine whether a renal nerve in the region was ablated by the heating based on assessment of a physiological parameter affected by ultrasound energy received at the renal nerve, for example as discussed above. The output module 308 can be configured to output an indicator of whether the region includes the target renal nerve and/or whether the renal nerve was ablated based on assessment of a physiological parameter affected by ultrasound energy received at the renal nerve. Each of the modules can provide control capabilities sufficient to perform the operations described herein or cause other components to perform such operations. The modules can be connected to, integral with, or include corresponding components, such as ultrasound device 50 and/or MRI device 150.

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

What is claimed is:

1. A method of performing and assessing a renal nerve ablation procedure, comprising:
stimulating a region with a first ultrasound energy from an ultrasound device;
based on an assessment, following initiation of the stimulating with the first ultrasound energy, of a physiological parameter affected by a target renal nerve, determining whether the region includes the target renal nerve;
when the region is determined to contain the target renal nerve, heating the region with a second ultrasound energy from the ultrasound device;
stimulating the region with a third ultrasound energy from the ultrasound device; and
based on an assessment, following initiation of the stimulating with the third ultrasound energy, of the physiological parameter, determining whether the target renal nerve was ablated.

2. The method of claim 1, further comprising, when the region is determined not to contain the target renal nerve, stimulating a different region with a fourth ultrasound energy from the ultrasound device.

3. The method of claim 1, further comprising:
measuring a first indicator of the physiological parameter prior to stimulating the region with the first ultrasound energy;
measuring a second indicator of the physiological parameter during and/or after the stimulating the region with the first ultrasound energy;
wherein determining whether the region includes the target renal nerve comprises comparing the first indicator to the second indicator.

4. The method of claim 1, further comprising:
measuring a first indicator of the physiological parameter during and/or after the stimulating the region with the first ultrasound energy;
measuring a second indicator of the physiological parameter during and/or after the stimulating the region with the third ultrasound energy;
wherein determining whether the target renal nerve was ablated comprises comparing the first indicator to the second indicator.

5. The method of claim 1, wherein the physiological parameter comprises blood pressure, renal blood flow rate, and/or a concentration of medulla norepinephrine in an anatomy of a patient.

6. The method of claim 1, further comprising, when the target renal nerve is determined to have been ablated, stimulating a different region with a fourth ultrasound energy from the ultrasound device.

7. The method of claim 1, wherein the first and/or third ultrasound energy satisfies an acoustic intensity threshold and/or a sonication pulse duration threshold necessary to achieve nerve stimulation.

8. The method of claim 7, wherein the acoustic intensity threshold is 0.1-100 W/cm² and the sonication pulse duration threshold is 5-250 ms.

9. The method of claim 1, wherein the first and/or third ultrasound energy does not satisfy an acoustic intensity threshold and/or a sonication pulse duration threshold necessary to achieve nerve ablation.

10. The method of claim 1, wherein the second ultrasound energy satisfies an acoustic intensity threshold and/or a sonication pulse duration threshold necessary to achieve nerve ablation.

11. A method, comprising:
stimulating a region with a first ultrasound energy from an ultrasound device;
based on an assessment, following initiation of the stimulating with the first ultrasound energy, of a physiological parameter affected by a target renal nerve, determining whether the region includes the target renal nerve;
when the region is determined to contain the target renal nerve, heating the region with a second ultrasound energy from the ultrasound device;
when the region is determined not to contain the target renal nerve, stimulating a different region with a third ultrasound energy from the ultrasound device.

12. The method of claim 11, further comprising:
measuring a first indicator of the physiological parameter prior to stimulating the region with the first ultrasound energy;
measuring a second indicator of the physiological parameter during and/or after the stimulating the region with the first ultrasound energy;
wherein determining whether the region includes the target renal nerve comprises comparing the first indicator to the second indicator.

13. The method of claim 11, wherein the physiological parameter comprises blood pressure, renal blood flow rate, and/or a concentration of medulla norepinephrine in an anatomy of a patient.

14. The method of claim 11, wherein the first and/or third ultrasound energy satisfies an acoustic intensity threshold and/or a sonication pulse duration threshold necessary to achieve nerve stimulation.

15. The method of claim 14, wherein the acoustic intensity threshold is 0.1-100 W/cm² and the sonication pulse duration threshold is 5-250 ms.

16. The method of claim 11, wherein the first and/or third ultrasound energy does not satisfy an acoustic intensity threshold and/or a sonication pulse duration threshold necessary to achieve nerve ablation.

17. The method of claim 11, wherein the second ultrasound energy satisfies an acoustic intensity threshold and/or a sonication pulse duration threshold necessary to achieve nerve ablation.

18. A method, comprising:
heating a region with a first ultrasound energy from an ultrasound device;
after the heating, stimulating the region with a second ultrasound energy from the ultrasound device; and
based on an assessment, following initiation of the stimulating with the second ultrasound energy, of a physiological parameter affected by a target renal nerve, determining whether a renal nerve in the region was ablated by the heating.

19. The method of claim 18, further comprising:
before heating the region, stimulating the region with a third ultrasound energy from the ultrasound device;
measuring a first indicator of the physiological parameter during and/or after the stimulating the region with the second ultrasound energy;
measuring a second indicator of the physiological parameter during and/or after the stimulating the region with the third ultrasound energy;
wherein determining whether the target renal nerve was ablated comprises comparing the first indicator to the second indicator.

20. The method of claim 18, further comprising, when the target renal nerve is determined to have been ablated, stimulating a different region with a third ultrasound energy from the ultrasound device.

21. The method of claim 18, further comprising, when the target renal nerve is determined not to have been ablated, further heating the region with a third ultrasound energy from the ultrasound device.

22. The method of claim 18, wherein the physiological parameter comprises blood pressure, renal blood flow rate, and/or a concentration of medulla norepinephrine in an anatomy of a patient.

23. The method of claim 18, wherein the second ultrasound energy satisfies an acoustic intensity threshold and/or a sonication pulse duration threshold necessary to achieve nerve stimulation.

24. The method of claim 23, wherein the acoustic intensity threshold is 0.1-100 W/cm² and the sonication pulse duration threshold is 5-250 ms.

25. The method of claim 18, wherein the second ultrasound energy does not satisfy an acoustic intensity threshold and/or a sonication pulse duration threshold necessary to achieve nerve ablation.

26. The method of claim 18, wherein the first ultrasound energy satisfies an acoustic intensity threshold and/or a sonication pulse duration threshold necessary to achieve nerve ablation.
